(19) United States
(12) Patent Application Publication
(10) Pub. No.: US 2025/0152755 A1
(43) Pub. Date: May 15, 2025

(54) SYSTEMS AND METHODS OF GENERATING LIPID, PROTEIN, AND/OR PROTEIN SHELLED BUBBLES

(71) Applicants: CASE WESTERN RESERVE UNIVERSITY, Cleveland, OH (US); UNIVERSITY HOSPITALS CLEVELAND MEDICAL CENTER, Cleveland, OH (US)

(72) Inventors: Agata Exner, Westlake, OH (US); Claire Counil, Cleveland, OH (US); Eric Abenojar, Cleveland, OH (US)

(21) Appl. No.: 18/727,234

(22) PCT Filed: Jan. 6, 2023
(86) PCT No.: PCT/US2023/060274

§ 371 Date: Jul. 8, 2024

Related U.S. Application Data
(60) Provisional application No. 63/297,014, filed on Jan. 6, 2022.
(60) Provisional application No. 63/336,481, filed on Apr. 29, 2022.

(51) Int. Cl. A61K 49/22; B01J 13/04

(52) U.S. Cl. CPC A61K 49/223; B01J 13/04

(57) ABSTRACT

A method of a generating a plurality of gas-core, lipid, protein and/or polymer shelled nanobubbles and/or microbubbles includes reversibly transferring a dispersion of at least one lipid, protein and/or polymer and a gas through a porous, e.g., microporous or nanoporous, membrane between a first depot and a second depot to provide a dispersion of lipid, protein and/or polymer shelled nanobubbles and/or microbubbles.

### RELATED APPLICATION

This application claims priority from U.S. Provisional Application No. 63/297,014, filed Jan. 6, 2022 and 63/336,481 filed Apr. 29, 2022, the subject matter of which are incorporated herein by reference in their entirety.

### GOVERNMENT FUNDING

This invention was made with government support under EB028144 awarded by The National Institutes of Health. The government has certain rights to the invention.

### BACKGROUND

Microbubble (MB) ultrasound contrast agents have been clinically utilized over the past >20 years. After the initial discoveries made nearly 50 years ago showing quickly-dissipating ultrasound signal generated from air bubbles in vivo several subsequent generations of MBs have improved their stability by adding a lipid, polymer, or protein shell and using a hydrophobic gas (such as perfluoropropane C₃F₈ or sulfur hexafluoride SF₆) for the core. Bubbles generate ultrasound contrast by oscillation in response to positive and negative pressure changes under an acoustic field. The response can vary according to the viscoelastisicty of the shell, the type of gas, the size of the bubbles and also the surrounding environment.

Lipid-shelled nanobubbles (NBs) with a fluorocarbon (FC) gas core are a promising new generation of theranostic agents for both ultrasound contrast imaging and controlled drug delivery applications. Clinically-used ultrasound contrast agents (UCAs) (1-10 μm MBs) are limited to intravascular applications while NBs, because of their size, can extravasate out of the vasculature and into the tumor tissue. This property makes it possible to use NBs for cancer theranostic applications, such as ultrasound image-guided surgeries and biopsies, tumor characterization, cell targeting, as well as ultrasound triggered and targeted drug delivery.

A formulation of NBs developed recently has led to improvement in NB stability in vitro and in vivo. These NBs are more resistant to deformation due to the incorporation of glycerol (Gly), a membrane stiffener known to increase buckling of lipid monolayers and addition of propylene glycol (PG), an edge-activator used in ultra-deformable liposomal formulations, which imparts flexibility to the NB shell. These components can be manipulated to obtain shells of varying viscoelastic properties, which can then be used to modulate bubble response in an acoustic field. For example, a recent publication described a pressure-dependent response of the NBs with three different shells. However, the effects were only apparent when the NBs population was filtered to reduce the polydispersity of NB size.

Size distribution is a known factor contributing to the bubble acoustic response. Reducing the polydispersity of bubbles results in more uniform nonlinear behavior and increased signal to noise ratio. The amplified activity increases the sensitivity of detection and bubble response at a specific pressure, which in turn, can lead to improvement in molecular imaging and drug delivery applications. These effects are predicted theoretically, and have been demonstrated experimentally, in vitro and in vivo. However, most current formulation methods used for production of NBs and MBs, do not yield a uniform bubble size. The most common techniques used are mechanical agitation, sonication and microfluidic assembly. For isolating NBs, a repurposed dental amalgamator (Vialmix) has been frequently used to force shell self-assembly around the gas core, followed by filtration or differential centrifugation to remove MBs. This process is inefficient and discards >50% of lipids utilized in the starting material. It also results in relatively polydisperse bubbles. Sonication also produces bubbles with a broad size distribution. While rapid MB assembly is effective with microfluidics, and new improvements have increased the production efficiency, the process is yet untested for NBs and requires specialized equipment.

### SUMMARY

Embodiments described herein relate to a system and method of generating a plurality of substantially monodisperse gas-core, lipid, protein and/or polymer shelled nanobubbles and/or microbubbles as well as apparatuses that can be employed in the system and method. The system and method are configured to pass a dispersion of lipid, protein and/or polymers (one or a mixture) multiple times through a porous membrane to arrive at a dispersion of monodispersed vesicle/liposome nanobubbles and/or microbubbles. The formation of the nanobubbles and/or microbubbles is made possible by extrusion of the dispersion of lipid, protein and/or polymers and a gas at a temperature higher than the phase transition temperature of the lipid, protein and/or polymers such that the lipid, protein and/or polymers are in a fluid state.

Advantageously, nanobubbles and/or microbubbles generated using the system and method described herein produce a yield that is at least 5 times higher in volume, with a similar quantity of bubbles when 49% less lipid, protein and/or polymers are used initially compared to nanobubbles and/or microbubbles generated using mechanical agitation (e.g., Vialmix) followed by differential centriguation. The extrusion method presented here also produced substantially smaller (about 160 nm vs about 320 nm) and more monodisperse or reduced polydispersity (full width at half maximum, FWHM) nanobubbles without drastically impacting the acoustical response of the nanobubbles. Additionally, we found that size, stabilty, concentration, and acoustic response of the nanobubbles and/or microbubbles can be modified or optimized by modifying or optimizing: (a) the temperature of extrusion, (b) the concentration of the lipid, protein and/or polymer solution, and (c) the number of passes through the extruder. Overall, the systems and methods describe herein can provide a simple, efficient and cost-effective nanobubble and/or microbubble production process, with potential for straightforward scale up. Moreover, testing of the nanobubbles and/or microbubbles generated by the system and method described herein in a biomedical imaging application showed feasibility of these nanobubbles and/or microbubbles as contrast agents with a strong and stable acoustic response, which is highly pressure dependent.

Accordingly, in some embodiments, a method of a generating a plurality of gas-core, lipid, protein, and/or polymer shelled nanobubbles and/or microbubbles can include reversibly transferring a dispersion of at least one lipid, protein and/or polymer and a gas through a porous, e.g., microporous or nanoporous, membrane between a first depot and a second depot to provide a dispersion of lipid, protein and/or polymer shelled nanobubbles and/or microbubbles. The dispersion of the at least one lipid, protein and/or polymer and gas can be reversibly transferred through the membrane using a first extruder and a second extruder that define, respectively, the first depot and the second depot.

The reversible transferring of the dispersion through the porous, e.g., microporous or nanoporous, membrane occurs at a temperature where the at least one lipid, protein and/or polymer is in a fluid state. The temperature can be higher than the solid to fluid phase transition temperature of the at least one lipid, protein and/or polymer, for example, about 37° C. to about 80° C., about 50° C. to about 75° C., or about 65° C.

In some embodiments, the first depot includes the dispersion of the at least one lipid, protein and/or polymer and gas, and the second depot includes a gas prior to reversible transferring of the dispersion through the membrane.

In some embodiments, the dispersion can be reversibly transferred through the microporous membrane about 2 to about 100 times, for example, about 10 to about 80 times, about 10 to about 70 times, about 10 to about 60 times, about 10 to about 50 times, or about 10 to about 50 times.

In other embodiments, the porous, e.g., microporous or nanoporous, membrane can have an average pore diameter of about 0.1 μm to about 2.0 μm, preferably, about 0.2 μm to about 1.5 μm, about 0.5 μm to about 1.2 μm, about 0.6 μm to about 1.0 μm, or about 0.8 μm.

In some embodiments, the method can further include optionally centrifuging the dispersion of lipid, protein and/or polymer shelled nanobubbles and/or microbubbles after reversibly transferring the dispersion of lipid, protein and/or polymer shelled nanobubbles and/or microbubbles through the porous, e.g., microporous or nanoporous, membrane to remove any foam.

In other embodiments, the method can further include passing the dispersion of lipid, protein and/or polymer shelled nanobubbles and/or microbubbles through a porous, e.g., microporous or nanoporous, filter after reversibly transferring or optionally centrifuging the dispersion of lipid, protein and/or polymer shelled nanobubbles and/or microbubbles. The porous, e.g., microporous or nanoporous, filter can have an average pore diameter similar to or less than the average pore diameter than the porous, e.g., microporous or nanoporous, membrane.

In some embodiments, the dispersion of the at least one lipid and gas includes a lipid solution. The lipid solution can have a lipid concentration of about 1 mg/ml to about 20 mg/ml, preferably, about 2 mg/ml to about 10 mg/ml, or about 5 mg/ml to about 7 mg/ml.

In some embodiments, the at least one lipid, protein and/or polymer can include a mixture of phospholipids having varying acyl chain lengths.

In some embodiments, the mixture of phospholipids can include at least two of dipalmitoylphosphatidylcholine (DPPC), dibehenoylglycerophosphocoline (DBPC), distearoylphosphatidylcholine (DSPC), diarachidonylphosphatidylcholine (DAPC), dioleoylphosphatidylethanolamine (DOPE), dipalmitoylphosphatidylethanolamine (DPPE), and distearoylphosphatidylethanolamine (DSPE); dipalmitoylphosphatidic acid (DPPA), or PEG functionalized lipids thereof.

In other embodiments, the mixture of phospholipids can include at least about 50% by weight of dibehenoylglycerophosphocoline (DBPC) and less than about 50% by weight of a combination of additional phospholipids selected from the group consisting of dipalmitoylphosphatidylcholine (DPPC), distearoylphosphatidylcholine (DSPC), diarachidonylphosphatidylcholine (DAPC), dioleoylphosphatidylethanolamine (DOPE), dipalmitoylphosphatidylethanolamine (DPPE), distearoylphosphatidylethanolamine (DSPE), dipalmitoylphosphatidic acid (DPPA), or PEG functionalized phospholipids thereof. For example, the mixture of phospholipids can include dibehenoylglycerophosphocoline (DBPC), dipalmitoylphosphatidic acid (DPPA), dipalmitoylphosphatidylethanolamine (DPPE), and distearoylphosphatidylethanolamine —N-methoxy-polyethylene glycol (DSPE-mPEG) at a ratio of about 6:1:2:1 by weight.

In some embodiments, the lipid solution can further include propylene glycol, glycerol, and phosphate buffered saline (PBS).

In some embodiments, the lipid solution consists essentially of dibehenoylglycerophosphocoline (DBPC), dipalmitoylphosphatidic acid (DPPA), dipalmitoylphosphatidylethanolamine (DPPE), and PEG functionalized distearoylphosphatidylethanolamine (DSPE), propylene glycol, glycerol, and phosphate buffered solution (PBS).

In some embodiments, the gas of the dispersion of the at least one lipid and gas includes a perfluorocarbon gas, such as C₃F₈, C₄F₁₀, C₅F₁₂, or other hydrophobic gasses such as sulfur hexafluoride or isofluorane, and/or other gasses such as oxygen, nitrogen, carbon dioxide or a mixture of gasses The gas of the dispersion in the first depot can be the same as the gas in the second depot.

In some embodiments, the nanobubbles can have a mean diameter of about 0.5 μm to about 0.4 μm, preferably, about 0.1 μm to about 0.3 μm or about 0.1 μm to about 0.2 μm.

In other embodiments, microbubbles can have a mean diameter of about 1 μm to about 4 μm, preferably, about 1 μm to about 3 μm or about 1 μm to about 2 μm.

Other embodiments described herein relate to a system for generating a plurality of gas-core, lipid, protein and/or polymer shelled nanobubbles and/or microbubbles. The system can include a first extruder configured to receive a dispersion of at least one lipid and gas, a second extruder configured to receive at least one gas, a channel configured to permit reversible fluid flow to and from the first extruder and the second extruder, and a porous, e.g., microporous or nanoporous, membrane provided in the channel. The first extruder and the second extruder are further configured to reversibly transfer the dispersion in first extruder and the gas in second extruder to and from the first extruder and second extruder through the channel and porous, e.g., microporous or nanoporous, membrane to provide a dispersion of lipid, protein and/or polymer shelled nanobubbles and/or microbubbles.

In some embodiments, the channel includes an outlet port configured to transfer the dispersion of lipid, protein and/or polymer shelled nanobubbles and/or microbubbles from the channel to a collection vessel.

In other embodiments, a first inlet port in fluid communication with the first extruder can be configured to receive the dispersion from a dispersion source. A second inlet port in fluid communication with the second extruder can be configured to receive the gas from a gas source.

In some embodiments, a first motor is configured to actuate the first extruder and a second motor is configured to the actuate the second extruder such that the dispersion and gas are reversibly transferred though the porous, e.g., microporous or nanoporous, membrane in the channel.

In some embodiments, the system can further include a heating module for maintaining the dispersion at a temperature where the at least one lipid is in a fluid state during reversible transfer of the dispersion and gas. For example, the heating module can be configured to maintain the temperature higher than the solid to fluid phase transition temperature of the at least one lipid, for example, about 37° C. to about 80° C., about 50° C. to about 75° C., or about 65° C.

In some embodiments, the porous, e.g., microporous or nanoporous, membrane can have an average pore diameter of about 0.1 μm to about 2.0 μm, for example, about 0.2 μm to about 1.5 μm, about 0.5 μm to about 1.2 μm, about 0.6 μm to about 1.0 μm, or about 0.8 μm.

In some embodiments, the system further includes a porous, e.g., microporous or nanoporous, filter configured to filter the dispersion of lipid, protein and/or polymer shelled nanobubbles and/or microbubbles after reversibly transferring the dispersion of lipid, protein and/or polymer shelled nanobubbles and/or microbubbles. The porous, e.g., microporous or nanoporous, filter can have an average pore diameter similar to or less than the average pore diameter than the porous, e.g., microporous or nanoporous, membrane.

In some embodiments, the dispersion of at least one lipid and gas can include a lipid solution. The lipid solution can have a lipid concentration of about 1 mg/ml to about 20 mg/m, for example, about 2 mg/ml to about 10 mg/ml, or about 5 mg/ml to about 7 mg/ml.

In some embodiments, the at least one lipid includes a mixture of phospholipids having varying acyl chain lengths. The mixture of phospholipids can include at least two of dipalmitoylphosphatidylcholine (DPPC), dibehenoylglycerophosphocoline (DBPC), distearoylphosphatidylcholine (DSPC), diarachidonylphosphatidylcholine (DAPC), dioleoylphosphatidylethanolamine (DOPE), dipalmitoylphosphatidylethanolamine (DPPE), and distearoylphosphatidylethanolamine (DSPE); dipalmitoylphosphatidic acid (DPPA), or PEG functionalized lipids thereof.

In some embodiments, the mixture of phospholipids includes at least about 50% by weight of dibehenoylglycerophosphocoline (DBPC) and less than about 50% by weight of a combination of additional phospholipids selected from the group consisting of dipalmitoylphosphatidylcholine (DPPC), distearoylphosphatidylcholine (DSPC), diarachidonylphosphatidylcholine (DAPC), dioleoylphosphatidylethanolamine (DOPE), dipalmitoylphosphatidylethanolamine (DPPE), distearoylphosphatidylethanolamine (DSPE), dipalmitoylphosphatidic acid (DPPA), or PEG functionalized phospholipids thereof. For example, the mixture of phospholipids can include dibehenoylglycerophosphocoline (DBPC), dipalmitoylphosphatidic acid (DPPA), dipalmitoylphosphatidylethanolamine (DPPE), and distearoylphosphatidylethanolamine —N-methoxy-polyethylene glycol (DSPE-mPEG) at a ratio of about 6:1:2:1 by weight.

In some embodiments, the lipid solution can further include propylene glycol, glycerol, and phosphate buffered solution (PBS). The lipid solution can consist essentially of dibehenoylglycerophosphocoline (DBPC), dipalmitoylphosphatidic acid (DPPA), dipalmitoylphosphatidylethanolamine (DPPE), and PEG functionalized distearoylphosphatidylethanolamine (DSPE), propylene glycol, glycerol, and phosphate buffered solution (PBS).

In some embodiments, the gas of the dispersion of the at least one lipid and gas can include a perfluorocarbon gas, such as C₃F₈.

Still other embodiments described herein relate to an apparatus for generating a plurality of gas-core, lipid, protein and/or polymer shelled nanobubbles and/or microbubbles. The apparatus comprises a housing that includes a first extruder configured to receive a dispersion of at least one lipid and gas, a second extruder configured to receive at least one gas, a channel configured to permit reversible fluid flow to and from the first extruder and the second extruded, and a porous, e.g., microporous or nanoporous, membrane provided in the channel, wherein the first extruder and the second extruder are further configured to reversibly transfer the dispersion in first extruder and the gas in second extruder to and from the first extruder and second extruder through the channel and porous, e.g., microporous or nanoporous, membrane to provide a dispersion of lipid, protein and/or polymer shelled nanobubbles and/or microbubbles.

In some embodiments, the channel includes an outlet port configured to transfer the dispersion of lipid, protein and/or polymer shelled nanobubbles and/or microbubbles from the channel and housing to a collection vessel.

In some embodiments, the apparatus further includes a first inlet port in fluid communication with the first extruder configured to receive the dispersion from a dispersion source, and a second inlet port in fluid communication with the second extruder configured to receive the gas from a gas source.

In some embodiments, the apparatus further includes a first motor contained within the housing configured to actuate the first extruder and a second motor contained within the housing configured to the actuate the second extruder such that the dispersion and gas are reversibly transferred though the porous, e.g., microporous or nanoporous, membrane in the channel.

In some embodiments, the first extruder includes a first plunger actuatable to move back and forth by the first motor, and the second extruder includes a second plunger actuatable to move back and forth by the second motor.

In some embodiments, the apparatus further includes a heat source contained within the housing for maintaining the dispersion at a temperature where the at least one lipid is in a fluid state during reversible transfer of the dispersion and gas. The heat source can be configured to maintain the temperature higher than the solid to fluid phase transition temperature of the at least one lipid, for example, about 37° C. to about 80° C., about 50° C. to about 75° C., or about 65° C.

In some embodiments, the porous, e.g., microporous or nanoporous, membrane can have an average pore diameter of about 0.1 μm to about 2.0 μm, for example, about 0.2 μm to about 1.5 μm, about 0.5 μm to about 1.2 μm, about 0.6 μm to about 1.0 μm, or about 0.8 μm.

In some embodiments, the outlet port includes a porous, e.g., microporous or nanoporous, filter configured to filter the dispersion of lipid, protein and/or polymer shelled nanobubbles and/or microbubbles after reversibly transferring the dispersion of lipid, protein and/or polymer shelled nanobubbles and/or microbubbles. The porous, e.g., microporous or nanoporous, filter has average pore diameter similar to or less than the average pore diameter than the porous, e.g., microporous or nanoporous, membrane.

### BRIEF DESCRIPTION OF THE DRAWINGS

FIG. 1 is flow diagram illustrating a method of generating a plurality of gas-core, lipid shelled nanobubbles and/or microbubbles.

FIG. 2 is an image and schematic illustration of a mini-extruder in accordance with an embodiment described herein.

FIG. 3 is a schematic illustration of an apparatus for generating a plurality of monodisperse gas-core, lipid shelled nanobubbles and/or microbubbles in accordance with one embodiment.

FIG. 4 is a schematic illustration of an apparatus for generating a plurality of monodisperse gas-core, lipid shelled nanobubbles and/or microbubbles in accordance with another embodiment.

FIGS. 5(A-C) illustrate physical characterization of standard self-assembly produced NBs (v-NBs) compared to the extruder-generated NBs (e-NBs) using resonant mass measurement: A. Buoyant and non-buoyant particle size and concentration distribution, B. Mean nanobubble diameter, and C. Total concentration of buoyant and non-buoyant particles. Asterisk indicates significant difference at p<0.05.

FIGS. 6(A-D) illustrate TEM images of (A) v-NBs and (C) e-NBs and distribution of size of (B) v-NBs and (D) e-NBs on 50 particles.

FIGS. 7(A-C) illustrate A. Preparation of the Lego®-based phantom used for acoustic evaluation of NBs and the US image acquisition set up including the three regions of interest which were measured to assess the pressure-dependent activity of NB solutions. The US focus is indicated by the blue arrow. B. Contrast harmonic images (12 MHz, MI: 0.22) at t=0 (left) and representation of the enhancement over 8 min (right) of v-NBs (B) and e-NBs (C). Panel A created with BioRender.

FIGS. 8(A-C) illustrate physical and acoustic characterization of the buoyant particle population of e-NBs prepared using different temperatures: A. Total concentration, B. mean diameter, and C. representation of enhancement of Z2 over 8 min as function of the temperature of extrusion, 50° C., 65° C., and 80° C. Asterisk indicates significant difference at p <0.05.

FIGS. 9(A-C) illustrate physical and acoustic characterization of the buoyant particle population of e-NBs prepared using different ratios of lipids and surfactants: A. Total concentration, B. mean diameter, and C. Acoustic response time intensity curve showing mean signal (line) and standard deviation of enhancement in Z2 region over 8 min (n=3). Asterisk indicates significant difference at p<0.05.

FIGS. 10(A-C) illustrate physical and acoustic characterization of the buoyant particle population of e-NBs prepared using different number of passes: A. Total concentration, B. mean diameter, and C. representation of enhancement of Z2 over 8 min as function of the pass number through the extruder, 10 (black), 20 (red), 30 (blue) and 40 (green). Asterisk indicates significant difference at p<0.05.

FIGS. 11(A-D) illustrate A. Representation of enhancement of liver (L) (A) and of kidney (K) (B) for v-NB and in vivo e-NBs over 30 min with a NB injection at 30 s (12 MHz, MI:0.2). Contrast harmonic images of in vivo e-NBs (C) and v-NBs (D) at t=lmin.

FIG. 12 is a schematic showing the preparation of nanobubbles using the mini-extruder. The extruder was outfitted with a 0.8 μm membrane. Syringes provided by Avanti are filled with lipid solution and FC gas, connected to the extruder and heated to the desired temperature on the heat block. For the extrusion, the mixture is passed 30 times through the extruder membrane. Following the extrusion process, the contents are emptied into a 15 mL tube, syringes are rinsed with the desired solvent, and all material is collected. The mixture is centrifuged for 2 min at 30 rcf and passed through a 0.45 μm PES filter. If preparing for imaging, the centrifugation step is omitted and the solution is passed through a 0.8 μm PES filter to remove foam. Created with BioRender.

FIGS. 13(A-E) illustrate physical characterization of v-NBs, e-NBs and in vivo e-NBs using resonant mass measurement: A. Buoyant particle size and concentration distribution, B. Total concentration of buoyant particles and C. Mean nanobubble diameter D. Contrast harmonic images (12 MHz, MI: 0.22) at t=0 of in vivo e-NBs. E. Representation of enhancement of in vivo e-NBs.

FIGS. 14(A-C) illustrate the percentage of the global enhancement for the upper zone (Z1), the focus zone (Z2) and the down zone (Z3) for e-NBs (A), for vivo e-NBs (B) and for v-NBs (C).

FIG. 15 illustrates TEM images of in vivo e-NBs.

FIGS. 16(A-B) illustrate percentage of the global enhancement of the kidney for the upper zone (orange), the focus zone (green) and the down zone (yellow) for v-NBs (A) and for in vivo e-NBs (B).

FIG. 17 illustrates MB removal system for e-NBs (left) and in vivo e-NBs (right). Created with BioRender.

### DETAILED DESCRIPTION

Unless defined otherwise, all technical and scientific terms used herein have the same meaning as commonly understood by one of ordinary skill in the art to which this invention belongs.

As used herein, each of the following terms has the meaning associated with it in this section.

As used herein and in the appended claims, the singular forms “a”, “an”, and “the” include plural references unless the context clearly dictates otherwise. Furthermore, the terms “a” (or “an”), “one or more” and “at least one” can be used interchangeably herein. The terms “comprising”, “including”, “having” and “constructed from” can also be used interchangeably.

“About” as used herein when referring to a measurable value such as an amount, a temporal duration, and the like, is meant to encompass variations of ±20%, ±10%, ±5%, ±1%, or ±0.1% from the specified value, as such variations are appropriate to perform the disclosed methods.

Ranges: throughout this disclosure, various aspects of the invention can be presented in a range format. It should be understood that the description in range format is merely for convenience and brevity and should not be construed as an inflexible limitation on the scope of the invention. Accordingly, the description of a range should be considered to have specifically disclosed all the possible subranges as well as individual numerical values within that range. For example, description of a range such as from 1 to 6 should be considered to have specifically disclosed subranges such as from 1 to 3, from 1 to 4, from 1 to 5, from 2 to 4, from 2 to 6, from 3 to 6 etc., as well as individual numbers within that range, for example, 1, 2, 2.7, 3, 4, 5, 5.3, and 6. This applies regardless of the breadth of the range.

Embodiments described herein relate to a system and method of a generating a plurality of gas-core, lipid, protein and/or polymer shelled nanobubbles and/or microbubbles as well as apparatuses that can be employed in the system and method. The system and method are configured to pass a dispersion of lipids (one or a mixture) multiple times through a porous membrane to arrive at a dispersion of monodispersed vesicle/liposome nanobubbles and/or microbubbles. The formation of the nanobubbles and/or microbubbles is made possible by extrusion of the dispersion of lipids and a gas at a temperature higher than the phase transition temperature of the lipids such that the lipids are in a fluid state.

Advantageously, nanobubbles and/or microbubbles generated using the system and method described herein produce a yield that is at least 5 times higher in volume, with a similar quantity of bubbles when 49% less lipids are used initially compared to nanobubbles and/or microbubbles generated using mechanical agitation (e.g., Vialmix) followed by differential centriguation. The extrusion method presented here also produced substantially smaller (about 160 nm vs about 320 nm) and more monodisperse or reduced polydispersity (full width at half maximum, FWHM) nanobubbles without drastically impacting the acoustical response of the nanobubbles. Additionally, we found that size, stabilty, concentration, and acoustic response of the nanobubbles and/or microbubbles can be modified or optimized by modifying or optimizing: (a) the temperature of extrusion, (b) the concentration of the lipid solution, and (c) the number of passes through the extruder. Overall, the systems and methods describe herein can provide a simple, efficient and cost-effective nanobubble and/or microbubble production process, with potential for straightforward scale up. Moreover, testing of the nanobubbles and/or microbubbles generated by the system and method described herein in a biomedical imaging application showed feasibility of these nanobubbles and/or microbubbles as contrast agents with a strong and stable acoustic response, which is highly pressure dependent.

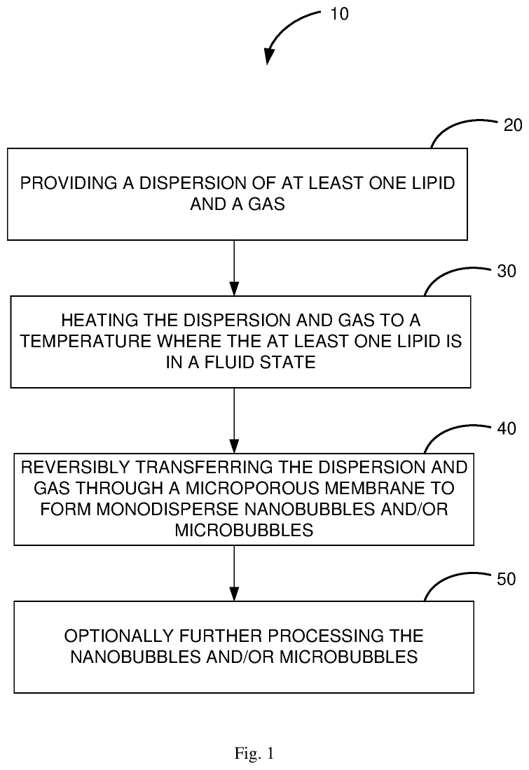

FIG. 1 is a flow diagram illustrating a method 10 of generating a plurality of gas-core, lipid, protein and/or polymer shelled nanobubbles and/or microbubbles in accordance with an embodiment described herein. The method at step 20 includes providing a dispersion of at least one lipid and a gas in a first depot and a gas in a second depot.

The dispersion of the at least one lipid and gas includes a lipid solution. The lipid solution include a plurality of lipids that are dissolved and/or mixed with an aqueous carrier, such as phosphate-buffered saline (PBS), and optionally an edge activator and/or membrane stiffener. The lipid solution can have a lipid concentration of at least about 2 mg/ml, at least about 3 mg/ml, at least about 4 mg/ml, at least about 5 mg/ml, about 6 mg/ml, at least about 7 mg/ml, at least about 8 mg/ml, at least about 9 mg/ml, at least about 10 mg/ml, at least about 11 mg/ml, at least about 12 mg/ml or more. In other embodiments, the lipid solution can have a lipid concentration of about 5 mg/ml to about 12 mg/ml, about 6 mg/ml to about 12 mg/ml, about 7 mg/ml to about 12 mg/ml, about 8 mg/ml to about 12 mg/ml, about 9 mg/ml to about 12 mg/ml, about 10 mg/ml to about 12 mg/ml, or at least about 10 mg/ml. In still other embodiments, the lipid concentration can be about 1 mg/ml to about 20 mg/ml, preferably, about 2 mg/ml to about 10 mg/ml, or about 5 mg/ml to about 7 mg/ml.

The at least one lipid provided in the lipid solution can include any naturally-occurring, synthetic or semi-synthetic (i.e., modified natural) moiety that is generally amphipathic or amphiphilic (i.e., including a hydrophilic component and a hydrophobic component). Examples of lipids, any one or combination of which may be used to form the nanobubbles and/or microbubbles, can include: phosphocholines, such as 1-alkyl-2-acetoyl-sn-glycero 3-phosphocholines, and 1-alkyl-2-hydroxy-sn-glycero 3-phosphocholines; phosphatidylcholine with both saturated and unsaturated lipids, including dioleoylphosphatidylcholine, dimyristoylphosphatidylcholine, dipentadecanoylphosphatidylcholine, dilauroylphosphatidylcholine, dipalmitoylphosphatidylcholine (DPPC), dibehenoylglycerophosphocoline (DBPC), distearoylphosphatidylcholine (DSPC), and diarachidonylphosphatidylcholine (DAPC); phosphatidylethanolamines, such as dioleoylphosphatidylethanolamine, dipalmitoylphosphatidylethanolamine (DPPE), and distearoylphosphatidylethanolamine (DSPE); phosphatidylserine; phosphatidylglycerols, including distearoylphosphatidylglycerol (DSPG); phosphatidylinositol; sphingolipids, such as sphingomyelin; glycolipids, such as ganglioside GM1 and GM2; glucolipids; sulfatides; glycosphingolipids; phosphatidic acids, such as dipalmitoylphosphatidic acid (DPPA) and distearoylphosphatidic acid (DSPA); palmitic acid; stearic acid; arachidonic acid; oleic acid; lipids bearing polymers, such as chitin, hyaluronic acid, polyvinylpyrrolidone or polyethylene glycol (PEG); lipids bearing sulfonated mono-, di-, oligo- or polysaccharides; cholesterol, cholesterol sulfate, and cholesterol hemisuccinate; tocopherol hemisuccinate; lipids with ether and ester-linked fatty acids; polymerized lipids (a wide variety of which are well known in the art); diacetyl phosphate; dicetyl phosphate; stearylaamine; cardiolipin; phospholipids with short chain fatty acids of about 6 to about 8 carbons in length; phospholipids with medium chain fatty acids of about 10 to about 16 carbons in length; phospholipids with long chain fatty acids of about 18 to about 24 carbons in length; synthetic phospholipids with asymmetric acyl chains, such as, for example, one acyl chain of about 6 carbons and another acyl chain of about 12 carbons; ceramides; non-ionic liposomes including niosomes, such as polyoxyalkylene (e.g., polyoxyethylene) fatty acid esters, polyoxyalkylene (e.g., polyoxyethylene) fatty alcohols, polyoxyalkylene (e.g., polyoxyethylene) fatty alcohol ethers, polyoxyalkylene (e.g., polyoxyethylene) sorbitan fatty acid esters (such as, for example, the class of compounds referred to as TWEEN (commercially available from ICI Americas, Inc., Wilmington, DE), glycerol polyethylene glycol oxystearate, glycerol polyethylene glycol ricinoleate, alkyloxylated (e.g., ethoxylated) soybean sterols, alkyloxylated (e.g., ethoxylated) castor oil, polyoxyethylene-polyoxypropylene polymers, and polyoxyalkylene (e.g., polyoxyethylene) fatty acid stearates; sterol aliphatic acid esters including cholesterol sulfate, cholesterol butyrate, cholesterol isobutyrate, cholesterol palmitate, cholesterol stearate, lanosterol acetate, ergosterol palmitate, and phytosterol n-butyrate; sterol esters of sugar acids including cholesterol glucuronide, lanosterol glucuronide, 7-dehydrocholesterol glucuronide, ergosterol glucuronide, cholesterol gluconate, lanosterol gluconate, and ergosterol gluconate; esters of sugar acids and alcohols including lauryl glucuronide, stearoyl glucuronide, myristoyl glucuronide, lauryl gluconate, myristoyl gluconate, and stearoyl gluconate; esters of sugars and aliphatic acids including sucrose laurate, fructose laurate, sucrose palmitate, sucrose stearate, glucuronic acid, gluconic acid and polyuronic acid; saponins including sarsasapogenin, smilagenin, hederagenin, oleanolic acid, and digitoxigenin; glycerol dilaurate, glycerol trilaurate, glycerol dipalmitate, glycerol and glycerol esters including glycerol tripalmitate, glycerol distearate, glycerol tristearate, glycerol dimyristate, glycerol trimyristate; long chain alcohols including n-decyl alcohol, lauryl alcohol, myristyl alcohol, cetyl alcohol, and n-octadecyl alcohol; 6-(5-cholesten-30-yloxy)-1-thio-β-D-galactopyranoside; digalactosyldiglyceride; 6-(5-cholesten-30-yloxy)hexyl-6-amino-6-deoxy-1-thio-β-D-galactopyranoside; 6-(5-cholesten-30-yloxy)hexyl-6-amino-6-deoxyl-1-thio-α-D-mannopyranoside; 12-(((7′-diethylaminocoumarin-3-yl)carbonyl)methylamino)octadecanoic acid; N-[12-(((7′-diethylaminocoumarin-3-yl)carbonyl)methylamino)octadecanoyl]-2-aminopalmitic acid; cholesteryl(4′-trimethylammonio)butanoate; N-succinyldioleoylphosphatidylethanolamine; 1,2-dioleoyl-sn-glycerol; 1,2-dipalmitoyl-sn-3-succinylglycerol; 1,3-dipalmitoyl-2-succinylglycerol; 1-hexadecyl-2-palmitoylglycerophosphoethanolamine and palmitoylhomocysteine; and/or any combinations thereof.

In some embodiments, the lipids provided in the lipid solution can include a mixture of phospholipids having varying acyl chain lengths. For example, the lipids can include a mixture of at least two of dipalmitoylphosphatidylcholine (DPPC), dibehenoylglycerophosphocoline (DBPC), distearoylphosphatidylcholine (DSPC), diarachidonylphosphatidylcholine (DAPC), dioleoylphosphatidylethanolamine (DOPE), dipalmitoylphosphatidylethanolamine (DPPE), and distearoylphosphatidylethanolamine (DSPE); dipalmitoylphosphatidic acid (DPPA), or PEG functionalized lipids thereof.

In other embodiments, the mixture of phospholipids having varying acyl chain length can include dibehenoylglycerophosphocoline (DBPC) and one or more additional phospholipids selected from the group consisting of dipalmitoylphosphatidylcholine (DPPC), distearoylphosphatidylcholine (DSPC), diarachidonylphosphatidylcholine (DAPC), dioleoylphosphatidylethanolamine (DOPE), dipalmitoylphosphatidylethanolamine (DPPE), distearoylphosphatidylethanolamine (DSPE), dipalmitoylphosphatidic acid (DPPA), or PEG functionalized phospholipids thereof.

In some embodiments, the mixture of phospholipids can include at least about 40%, at least about 50%, at least about 60%, at least about 70%, or at about 80%, by weight of dibehenoylglycerophosphocoline (DBPC); and less than about 60%, less than about 50%, less than about 40%, less than about 30%, or less than about 20%, by weight, of a combination of additional phospholipids selected from the group consisting of dipalmitoylphosphatidylcholine (DPPC), distearoylphosphatidylcholine (DSPC), diarachidonylphosphatidylcholine (DAPC), dioleoylphosphatidylethanolamine (DOPE), dipalmitoylphosphatidylethanolamine (DPPE), distearoylphosphatidylethanolamine (DSPE), dipalmitoylphosphatidic acid (DPPA), or PEG functionalized phospholipids thereof. The PEG can have a molecular weight of about 1000 to about 5000 Da, for example, about 2000 Da.

In some embodiments, the mixture of phospholipids can include about 40% to about 80%, about 50% to about 70%, or about 55% to about 65% (e.g., about 60%) by weight dibehenoylglycerophosphocoline (DBPC); and about 20% to about 60%, about 30% to about 50%, or about 35% to about 45% (e.g., about 40%) by weight of a combination of additional phospholipids selected from the group consisting of dipalmitoylphosphatidylcholine (DPPC), distearoylphosphatidylcholine (DSPC), diarachidonylphosphatidylcholine (DAPC), dioleoylphosphatidylethanolamine (DOPE), dipalmitoylphosphatidylethanolamine (DPPE), distearoylphosphatidylethanolamine (DSPE), dipalmitoylphosphatidic acid (DPPA), or PEG functionalized phospholipids thereof.

In other embodiments, the one or more additional phospholipids can include, consist essentially of, or consists of a combination of dipalmitoylphosphatidic acid (DPPA), dipalmitoylphosphatidylethanolamine (DPPE), and PEG functionalized distearoylphosphatidylethanolamine (DSPE).

In still other embodiments, the mixture of phospholipids can include dibehenoylglycerophosphocoline (DBPC), dipalmitoylphosphatidic acid (DPPA), dipalmitoylphosphatidylethanolamine (DPPE), and PEG functionalized distearoylphosphatidylethanolamine (DSPE) at a ratio of, for example, about 6:1:1:1 by weight.

The edge activator can enhance the flexibility of the nanobubbles and/or microbubbles formed using the method described herein. The edge activator can include a co-surfactant, such as propylene glycol, which enhances the effectiveness of phospholipid surfactants. The edge activator can be provided in the lipid solution at an amount effective to cause separation of lipid domains during nanobubble and/or microbubble formation and form defects in the nanobubble and/or microbubble that absorb excessive pressure, which could have caused lipid “domain” tearing. Other edge activators, which can be substituted for propylene glycol or used in combination with propylene glycol, can include cholesterol, sodium cholate, limonene, oleic acid, and/or span 80.

In some embodiments an amount of propylene glycol provided in the lipid solution can be about 0.05 ml to about 0.5 ml, about 0.06 ml to about 0.4 ml, about 0.07 ml to about 0.3 ml, about 0.08 ml to about 0.2 ml, or about 0.1 ml, per 1 ml of hydrated lipids.

An example of a membrane stiffener that can be provide in the lipid solution is glycerol. Glycerol can be provided in the lipid solution at an amount effective to stiffen the nanobubbles and/or microbubbles and inhibit lipid “domain” tearing. The amount of glycerol provided in the lipid solution can be about 0.05 ml to about 0.5 ml, about 0.06 ml to about 0.4 ml, about 0.07 ml to about 0.3 ml, about 0.08 ml to about 0.2 ml, or about 0.1 ml, per 1 ml of hydrated lipids.

In one example, the lipid solution can be prepared by dissolving a plurality of lipids, such as DBPC, DPPA, DPPE, and mPEG-DSPE, in propylene glycol. A mixture of glycerol and phosphate-buffered saline (PBS) can then be added to the lipid solution after dissolution of the plurality lipids at temperature above the phase transition temperature of the lipids, e.g., about 80° C. This lipid solution can then be diluted with a mixture of propylene glycol, glycerol, and PBS to provide a dilute lipid solution having concentration of lipids as described herein.

In some embodiments, the lipid solution can also include other materials, such as liquids, oils, bioactive agents, diagnostic agents, photoacoustic agents, and/or therapeutic agents. The materials can be encapsulated by the nanobubbles and/or microbubbles formed from the lipid solution.

Bioactive agents e can include any substance capable of exerting a biological effect in vitro and/or in vivo. Examples of bioactive agents can include, but are not limited to, chemotherapeutic agents, biologically active ligands, small molecules, DNA fragments, DNA plasmids, interfering RNA molecules, such as siRNAs, oligonucleotides, and DNA encoding for shRNA. Diagnostic agents can include any substance that may be used for imaging a region of interest (ROI) in a subject and/or diagnosing the presence or absence of a disease or diseased tissue in a subject. Therapeutic agents can refer to any therapeutic or prophylactic agent used in the treatment (including the prevention, diagnosis, alleviation, or cure) of a malady, affliction, condition, disease or injury in a subject. It will be appreciated that the membrane can additionally or optionally include proteins, carbohydrates, polymers, surfactants, and/or other membrane stabilizing materials, any one or combination of which may be natural, synthetic, or semi-synthetic.

In some embodiments, the bioactive agent can include a therapeutic agent, such as a chemotherapeutic agent, an anti-proliferative agent, an anti-microbial agent, a biocidal agent, and/or a biostatic agent. The therapeutic agent can be encapsulated by and/or linked to the membrane of the nanobubble.

In some embodiments, the lipid solution can additionally or optionally include at least one targeting moiety that is capable of targeting and/or adhering the nanobubble to a cell or tissue of interest. In some embodiments, the targeting moiety can comprise any molecule, or complex of molecules, which is/are capable of interacting with an intracellular, cell surface, or extracellular biomarker of the cell. The biomarker can include, for example, a cellular protease, a kinase, a protein, a cell surface receptor, a lipid, and/or fatty acid. Other examples of biomarkers that the targeting moiety can interact with include molecules associated with a particular disease. For example, the biomarkers can include cell surface receptors implicated in cancer development, such as CA-125 receptor, epidermal growth factor receptor, and transferrin receptor. The targeting moiety can interact with the biomarkers through non-covalent binding, covalent binding, hydrogen binding, van der Waals forces, ionic bonds, hydrophobic interactions, electrostatic interaction, and/or combinations thereof.

The targeting moiety can include, but is not limited to, synthetic compounds, natural compounds or products, macromolecular entities, bioengineered molecules (e.g., polypeptides, lipids, polynucleotides, antibodies, antibody fragments), and small entities (e.g., small molecules, neurotransmitters, substrates, ligands, hormones and elemental compounds).

In one example, the targeting moiety can comprise an antibody, such as a monoclonal antibody, a polyclonal antibody, or a humanized antibody, including without limitation: Fv fragments, single chain Fv (scFv) fragments, Fab′ fragments, F(ab′)2 fragments, single domain antibodies, camelized antibodies and antibody fragments, humanized antibodies and antibody fragments, and multivalent versions of the foregoing; multivalent targeting moieties including without limitation: monospecific or bispecific antibodies, such as disulfide stabilized Fv fragments, scFv tandems ((scFv)₂ fragments), diabodies, tribodies or tetrabodies, which typically are covalently linked or otherwise stabilized (i.e., leucine zipper or helix stabilized) scFv fragments; and receptor molecules, which naturally interact with a desired target molecule.

Preparation of antibodies may be accomplished by any number of well-known methods for generating antibodies. These methods typically include the step of immunization of animals, typically mice, with a desired immunogen (e.g., a desired target molecule or fragment thereof). Once the mice have been immunized, and boosted one or more times with the desired immunogen(s), antibody-producing hybridomas may be prepared and screened according to well-known methods. See, for example, Kuby, Janis, Immunology, Third Edition, pp. 131-139, W.H. Freeman & Co. (1997), for a general overview of monoclonal antibody production, that portion of which is incorporated herein by reference.

The targeting moiety need not originate from a biological source. The targeting moiety may, for example, be screened from a combinatorial library of synthetic peptides. One such method is described in U.S. Pat. No. 5,948,635, incorporated herein by reference, which describes the production of phagemid libraries having random amino acid insertions in the pIII gene of M13. This phage may be clonally amplified by affinity selection.

The immunogens used to prepare targeting moieties having a desired specificity will generally be the target molecule, or a fragment or derivative thereof. Such immunogens may be isolated from a source where they are naturally occurring or may be synthesized using methods known in the art. For example, peptide chains may be synthesized by 1-ethyl-3-[dimethylaminoproply]carbodiimide (EDC)-catalyzed condensation of amine and carboxyl groups. In certain embodiments, the immunogen may be linked to a carrier bead or protein. For example, the carrier may be a functionalized bead such as SASRIN resin commercially available from Bachem, King of Prussia, Pa. or a protein such as keyhole limpet hemocyanin (KLH) or bovine serum albumin (BSA). The immunogen may be attached directly to the carrier or may be associated with the carrier via a linker, such as a non-immunogenic synthetic linker (for example, a polyethylene glycol (PEG) residue, amino caproic acid or derivatives thereof) or a random, or semi-random polypeptide.

In certain embodiments, it may be desirable to mutate the binding region of the polypeptide targeting moiety and select for a targeting moiety with superior binding characteristics as compared to the un-mutated targeting moiety. This may be accomplished by any standard mutagenesis technique, such as by PCR with Taq polymerase under conditions that cause errors. In such a case, the PCR primers could be used to amplify scFv-encoding sequences of phagemid plasmids under conditions that would cause mutations. The PCR product may then be cloned into a phagemid vector and screened for the desired specificity, as described above.

In other embodiments, the targeting moiety may be modified to make them more resistant to cleavage by proteases. For example, the stability of targeting moiety comprising a polypeptide may be increased by substituting one or more of the naturally occurring amino acids in the (L) configuration with D-amino acids. In various embodiments, at least 1%, 5%, 10%, 20%, 50%, 80%, 90% or 100% of the amino acid residues of targeting moiety may be of the D configuration. The switch from L to D amino acids neutralizes the digestion capabilities of many of the ubiquitous peptidases found in the digestive tract. Alternatively, enhanced stability of a targeting moiety comprising a peptide bond may be achieved by the introduction of modifications of the traditional peptide linkages. For example, the introduction of a cyclic ring within the polypeptide backbone may confer enhanced stability in order to circumvent the effect of many proteolytic enzymes known to digest polypeptides in the stomach or other digestive organs and in serum. In still other embodiments, enhanced stability of a targeting moiety may be achieved by intercalating one or more dextrorotatory amino acids (such as, dextrorotatory phenylalanine or dextrorotatory tryptophan) between the amino acids of targeting moiety. In exemplary embodiments, such modifications increase the protease resistance of a targeting moiety without affecting the activity or specificity of the interaction with a desired target molecule.

In certain embodiments, the antibodies or variants thereof may be modified to make them less immunogenic when administered to a subject. For example, if the subject is human, the antibody may be “humanized”; where the complimentarily determining region(s) of the hybridoma-derived antibody has been transplanted into a human monoclonal antibody, for example as described in Jones, P. et al. (1986), Nature, 321, 522-525 or Tempest et al. (1991), Biotechnology, 9, 266-273. Also, transgenic mice, or other mammals, may be used to express humanized antibodies. Such humanization may be partial or complete.

In certain embodiments, a targeting moiety as described herein may comprise a homing peptide, which selectively directs the nanobubble to a targeted cell. Homing peptides for a targeted cell can be identified using various methods well known in the art. Many laboratories have identified the homing peptides that are selective for cells of the vasculature of brain, kidney, lung, skin, pancreas, intestine, uterus, adrenal gland, retina, muscle, prostate, or tumors. See, for example, Samoylova et al., 1999, Muscle Nerve, 22:460; Pasqualini et al., 1996 Nature, 380:364; Koivunen et al., 1995, Biotechnology, 13:265; Pasqualini et al., 1995, J. Cell Biol., 130:1189; Pasqualini et al., 1996, Mole. Psych., 1:421, 423; Rajotte et al., 1998, J. Clin. Invest., 102:430; Rajotte et al., 1999, J. Biol. Chem., 274:11593. See, also, U.S. Pat. Nos. 5,622,6999; 6,068,829; 6,174,687; 6,180,084; 6,232,287; 6,296,832; 6,303,573; and 6,306,365.

Phage display technology provides a means for expressing a diverse population of random or selectively randomized peptides. Various methods of phage display and methods for producing diverse populations of peptides are well known in the art. For example, methods for preparing diverse populations of binding domains on the surface of a phage have been described in U.S. Pat. No. 5,223,409. In particular, phage vectors useful for producing a phage display library as well as methods for selecting potential binding domains and producing randomly or selectively mutated binding domains are also provided in U.S. Pat. No. 5,223,409. Similarly, methods of producing phage peptide display libraries, including vectors and methods of diversifying the population of peptides that are expressed, are also described in Smith et al., 1993, Meth. Enzymol., 217:228-257, Scott et al., Science, 249:386-390, and two PCT publications WO 91/07141 and WO 91/07149. Phage display technology can be particularly powerful when used, for example, with a codon based mutagenesis method, which can be used to produce random peptides or randomly or desirably biased peptides (see, e.g., U.S. Pat. No. 5,264,563). These or other well-known methods can be used to produce a phage display library, which can be subjected to the in vivo phage display method in order to identify a peptide that homes to one or a few selected tissues.

In vitro screening of phage libraries has previously been used to identify peptides that bind to antibodies or cell surface receptors (see, e.g., Smith, et al., 1993, Meth. Enzymol., 217:228-257). For example, in vitro screening of phage peptide display libraries has been used to identify novel peptides that specifically bind to integrin adhesion receptors (see, e.g., Koivunen et al., 1994, J. Cell Biol. 124:373-380), and to the human urokinase receptor (Goodson, et al., 1994, Proc. Natl. Acad. Sci., USA 91:7129-7133).

In certain embodiments, the targeting moiety may comprise a receptor molecule, including, for example, receptors, which naturally recognize a specific desired molecule of a target cell. Such receptor molecules include receptors that have been modified to increase their specificity of interaction with a target molecule, receptors that have been modified to interact with a desired target molecule not naturally recognized by the receptor, and fragments of such receptors (see, e.g., Skerra, 2000, J. Molecular Recognition, 13:167-187). A preferred receptor is a chemokine receptor. Exemplary chemokine receptors have been described in, for example, Lapidot et al, 2002, Exp Hematol, 30:973-81 and Onuffer et al, 2002, Trends Pharmacol Sci, 23:459-67.

In other embodiments, the targeting moiety may comprise a ligand molecule, including, for example, ligands which naturally recognize a specific desired receptor of a target cell. Such ligand molecules include ligands that have been modified to increase their specificity of interaction with a target receptor, ligands that have been modified to interact with a desired receptor not naturally recognized by the ligand, and fragments of such ligands.

In still other embodiments, the targeting moiety may comprise an aptamer. Aptamers are oligonucleotides that are selected to bind specifically to a desired molecular structure of the target cell. Aptamers typically are the products of an affinity selection process similar to the affinity selection of phage display (also known as in vitro molecular evolution). The process involves performing several tandem iterations of affinity separation, e.g., using a solid support to which the diseased immunogen is bound, followed by polymerase chain reaction (PCR) to amplify nucleic acids that bound to the immunogens. Each round of affinity separation thus enriches the nucleic acid population for molecules that successfully bind the desired immunogen. In this manner, a random pool of nucleic acids may be “educated” to yield aptamers that specifically bind target molecules. Aptamers typically are RNA, but may be DNA or analogs or derivatives thereof, such as, without limitation, peptide nucleic acids (PNAs) and phosphorothioate nucleic acids.

In yet other embodiments, the targeting moiety may be a peptidomimetic. By employing, for example, scanning mutagenesis to map the amino acid residues of a protein, which is involved in binding other proteins, peptidomimetic compounds can be generated that mimic those residues, which facilitate the interaction. Such mimetics may then be used as a targeting moiety to deliver the nanobubble to a target cell. For instance, non-hydrolyzable peptide analogs of such resides can be generated using benzodiazepine (e.g., see Freidinger et al. in Peptides: Chemisty and Biology, G. R. Marshall ed., ESCOM Publisher: Leiden, Netherlands, 1988), azepine (e.g., see Huffman et al. in Peptides: Chemistry and Biology, G. R. Marshall ed., ESCOM Publisher: Leiden, Netherlands, 1988), substituted gamma lactam rings (Garvey et al. in Peptides: Chemistry and Biology, G. R. Marshall ed., ESCOM Publisher: Leiden, Netherlands, 1988), keto-methylene pseudopeptides (Ewenson et al., 1986, J Med Chem 29:295; and Ewenson et al., in Peptides: Structure and Function (Proceedings of the 9th American Peptide Symposium) Pierce Chemical Co. Rockland, Ill., 1985), b-turn dipeptide cores (Nagai et al., 1985, Tetrahedron Lett 26:647; and Sato et al., 1986, J Chem Soc Perkin Trans 1:1231), and β-aminoalcohols (Gordon et al., 1985, Biochem Biophys Res Cummun 126:419; and Dann et al., 1986, Biochem Biophys Res Commun 134:71).

The lipid solution can be provided in a vial with at least one gas. Advantageously, the gas can have a low solubility in water. A gas that has a low solubility in water can include, for example, hexafluoro acetone; isopropyl acetylene; allene; tetrafluoroallene; boron trifluoride; 1,2-butadiene; 1,3-butadiene; 1,3-butadiene; 1,2,3-trichloro, 2-fluoro-1,3-butadiene; 2-methyl,1,3 butadiene; hexafluoro-1,3-butadiene; butadiyne; 1-fluoro-butane; 2-methyl-butane; decafluoro butane; 1-butene; 2-butene; 2-methy-1-butene; 3-methyl-1-butene; perfluoro-1-butene; perfluoro-1-butene; perfluoro-2-butene; 1,4-phenyl-3-butene-2-one; 2-methyl-1-butene-3-yne; butyl nitrate; 1-butyne; 2-butyne; 2-chloro-1,1,1,4,4,4-hexafluoro-butyne; 3-methyl-1-butyne; perfluoro-2-butyne; 2-bromo-butyraldehyde; carbonyl sulfide; crotononitrile; cyclobutane; methyl-cyclobutane; octafluoro-cyclobutane; perfluoro-cyclobutene; 3-chloro-cyclopentene; perfluoro ethane; perfluoro propane; perfluoro butane; perfluoro pentane; perfluoro hexane; cyclopropane; 1,2-dimethyl-cyclopropane; 1,1-dimethyl cyclopropane; 1,2-dimethyl cyclopropane; ethyl cyclopropane; methyl cyclopropane; diacetylene; 3-ethyl-3-methyl diaziridine; 1,1,1-trifluorodiazoethane; dimethyl amine; hexafluorodimethyl amine; dimethylethylamine; -bis-(Dimethyl phosphine)amine; 2,3-dimethyl-2-norbornane; perfluorodimethylamine; dimethyloxonium chloride; 1,3-dioxolane-2-one; perfluorocarbons such as and not limited to 4-methyl,1,1,1,2-tetrafluoro ethane; 1,1,1-trifluoroethane; 1,1,2,2-tetrafluoroethane; 1,1,2-trichloro-1,2,2-trifluoroethane; 1,1 dichloroethane; 1,1-dichloro-1,2,2,2-tetrafluoro ethane; 1,2-difluoro ethane; 1-chloro-1,1,2,2,2-pentafluoro ethane; 2-chloro, 1,1-difluoroethane; 1-chloro-1,1,2,2-tetrafluoro ethane; 2-chloro, 1,1-difluoroethane; chloroethane; chloropentafluoro ethane; dichlorotrifluoroethane; fluoro-ethane; hexafluoro-ethane; nitro-pentafluoro ethane; nitroso-pentafluoro ethane; perfluoro ethane; perfluoro ethylamine; ethyl vinyl ether; 1,1-dichloro ethylene; 1,1-dichloro-1,2-difluoro ethylene; 1,2-difluoro ethylene; Methane; Methane-sulfonyl chloride-trifluoro; Methanesulfonyl fluoride-trifluoro; Methane-(pentafluorothio)trifluoro; Methane-bromo difluoro nitroso; Methane-bromo fluoro; Methane-bromo chloro-fluoro; Methanebromo-trifluoro; Methane-chloro difluoro nitro; Methane-chloro dinitro; Methanechloro fluoro; Methane-chloro trifluoro; Methane-chloro-difluoro; Methane dibromo difluoro; Methane-dichloro difluoro; Methane-dichloro-fluoro; Methanedifluoro; Methane-difluoro-iodo; Methane-disilano; Methane-fluoro; Methaneiodo; Methane-iodo-trifluoro; Methane-nitro-trifluoro; Methane-nitroso-trifluoro; Methane-tetrafluoro; Methane-trichlorofluoro; Methane-trifluoro; Methanesulfenylchloride-trifluoro; 2-Methyl butane; Methyl ether; Methyl isopropyl ether; Methyl lactate; Methyl nitrite; Methyl sulfide; Methyl vinyl ether; Neon; Neopentane; Nitrogen (N.sub.2); Nitrous oxide; 1,2,3-Nonadecane tricarboxylic acid-2-hydroxytrimethylester; 1-Nonene-3-yne; Oxygen (O₂); 1, 4-Pentadiene; n-Pentane; Pentane-perfluoro; 2-Pentanone-4-amino-4-methyl; 1-Pentene; 2-Pentene [cis]; 2-Pentene (trans); 1-Pentene-3-bromo; 1-Pentene-perfluoro; Phthalic acid-tetrachloro; Piperidine-2,3,6-trimethyl; Propane, Propane-1,1,1,2,2,3-hexafluoro; Propane-1,2-epoxy; Propane-2,2 difluoro; Propane 2-amino, Propane-2-chloro; Propane-heptafluoro-1-nitro; Propane-heptafluoro-1-nitroso; Propane-perfluoro; Propene; Propyl-1,1,1,2,3,3-hexafluoro-2,3 dichloro; Propylene-1-chloro; Propylenechloro-(trans); Propylene-2-chloro; Propylene-3-fluoro; Propylene-perfluoro; Propyne; Propyne-3,3,3-trifluoro; Styrene-3-fluoro; Sulfur hexafluoride; Sulfur (di)-decafluoro(S2F10); Toluene-2,4-diamino; Trifluoroacetonitrile; Trifluoromethyl peroxide; Trifluoromethyl sulfide; Tungsten hexafluoride; Vinyl acetylene; Vinyl ether; Xenon; Nitrogen; air; carbon dioxide, nitric oxide, and other ambient gases.

In some embodiments, the gas is a perfluorocarbon. Perfluorocarbons can include, for example, perfluoromethane, perfluoroethane, perfluoropropane, perfluorobutane, perfluoropentane, perfluorohexane. In one example, the perfluorocarbon gas is octafluoropropane (C₃F₈).

The gas provided in the second depot can be the same as the gas in the dispersion provided in the first depot. In some embodiment, the gas provided in the first depot and the second depot can be a perfluorocarbon, such as perfluoromethane, perfluoroethane, perfluoropropane, perfluorobutane, perfluoropentane, perfluorohexane, preferably, octafluoropropane (C₃F₈).

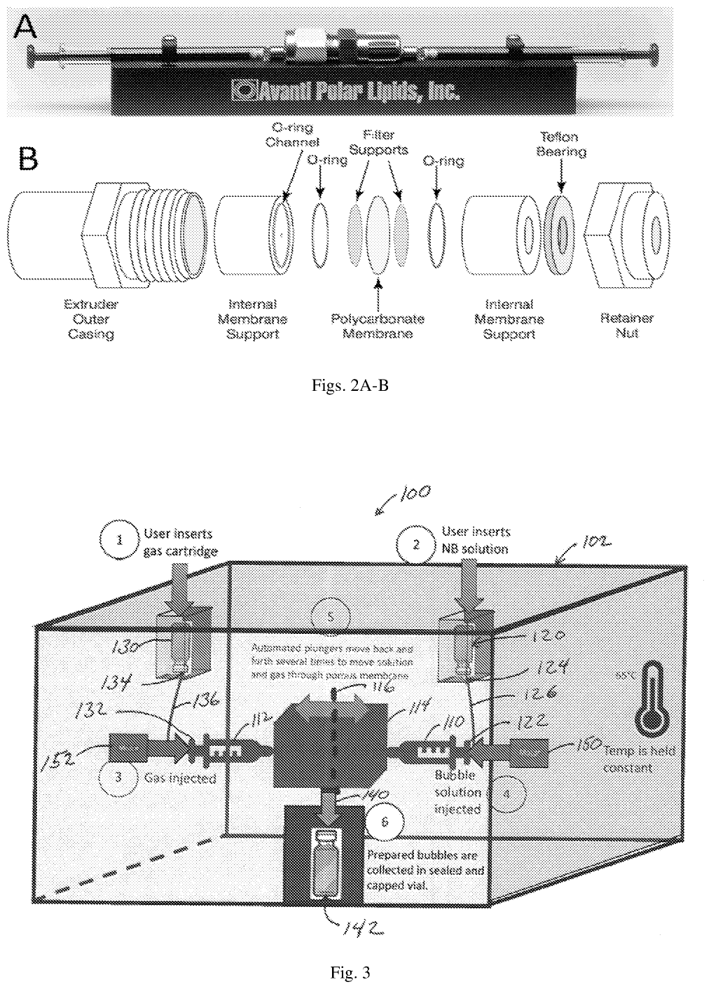

As illustrated in FIG. 2, the first depot and the second depot can include a first extruder and a second extruder. The first extruder is configured to receive the dispersion of the at least one lipid and gas, and the second extruder is configured to receive at least one gas. The first extruder and second extruder are fluidly connected by a channel that permits reversible fluid flow of the dispersion and gas to and from the first extruder and the second extruder upon actuation. The channel includes a porous, e.g., microporous or nanoporous, membrane provided in the channel such that fluid flowing through channel to and from the first extruder and second extruder passes through the porous, e.g., microporous or nanoporous, membrane in the channel. The porous, e.g., microporous or nanoporous, membrane can have an average pore diameter of about 0.1 μm to about 2.0 μm, preferably, about 0.2 μm to about 1.5 μm, about 0.5 μm to about 1.2 μm, about 0.6 μm to about 1.0 μm, or about 0.8 μm.

The first extruder and the second extruder are further configured to reversibly transfer the dispersion in first extruder and the gas in second extruder to and from the first extruder and second extruder through the channel and porous, e.g., microporous or nanoporous, membrane.

Referring again to FIG. 1, at step 30, the dispersion in the first depot or first extruder is heated to a temperature where the at least one lipid is in a fluid state. The temperature can be higher than the solid to fluid phase transition temperature of the at least one lipid, for example, about 37° C. to about 80° C., about 50° C. to about 75° C., or about 65° C.

At step 40, the dispersion of the at least one lipid and the gas can be reversibly transferred through the porous, e.g., microporous or nanoporous, membrane between the first extruder and the second extruder to generate a dispersion of lipid, protein and/or polymer shelled nanobubbles and/or microbubbles. The reversible transferring of the dispersion through the porous, e.g., microporous or nanoporous, membrane occurs at a temperature where the at least one lipid is in a fluid state. The temperature can be higher than the solid to fluid phase transition temperature of the at least one lipid, for example, about 37° C. to about 80° C., about 50° C. to about 75° C., or about 65° C.

Advantageously, the temperature of the dispersion during reversible transfer through the porous, e.g., microporous or nanoporous, membrane can be controlled to modify or optimize the size, concentration, acoustic properties of the nanobubble and/or microbubbles formed during extrusion while mitigating formation of non-buoyant particles. Temperature affects the fluidity of the solution and allows the dispersion of the lipid solution and gas to pass between the first extruder and the second extruder without producing liquid-core self-assembly devoid of a gas. A temperature substantially higher than the transition phase of the lipid, for example, greater than 80° C., will result in the formation of more non buoyant particles. On the contrary, if the temperature is too low, for example, at 50° C., the lipids are not fluid enough to easily pass through the porous, e.g., microporous or nanoporous, membrane.

As discussed in the Example below, a dispersion of a lipid solution and gas was reversibly transferred through the porous, e.g., microporous or nanoporous, membrane at 50, 65 and 80° C. to determine the optimal parameter for formulation of buoyant nanobubbles and the effect of temperature on nanobubble size, yield and ultrasound signal intensity was assessed. At 65° C. resulting nanobubbles formed by the method described herein had the smallest diameter, highest concentration and strong acoustic activity. Further increasing the temperature to 80° C. resulted in a decreased bubble concentration and a significant drop in acoustic activity. This data suggests that 80° C. is ideal to form nonbuoyant particles because it is higher than the transition temperature of the phospholipids used in the formulation. Lowering the temperature to 50° C. from 65° C., also resulted in a decrease in nanobubble concentration due to a reduction in the fluidity of the phospholipid solution, but an increase in bubble diameter. Reducing the fluidity of the lipid solution, in turn, reduces the efficiency of the formation of foam and consequently, the formation of the nanobubbles.

The dispersion can be reversibly transferred through the porous, e.g., microporous or nanoporous, membrane for a number passes effective to form the nanobubbles and/or microbubbles. In some embodiments, the number of passes can be about 2 to about 100, for example, about 10 to about 80, about 10 to about 70, about 10 to about 60, about 10 to about 50, or about 10 to about 50 to form the nanobubbles and/or microbubbles.

The number of passes through the porous, e.g., microporous or nanoporous, membrane and extruders can influence the polydispersity of resulting nanobubbles and/or microbubble. An increase in the number of times the dispersion of lipid solution passes through the porous, e.g., microporous or nanoporous, membrane between the extruders typically leads to more monodisperse nanobubbles and/or microbubbles. As discussed in the Example, we examined a range of 10 to 40 passes. The concentration slightly increased comparing 10-pass to 30-pass samples from 2.5±0.5×10¹⁰ to 6.2±1.8×10¹⁰ nanobubbles mL⁻¹ and then decreased for the 40-pass samples. This suggests that that the majority of nanobubbles are formed in the first steps of the reversible transfer process. This was also confirmed by comparable acoustic response for the different number of passes used.

The substantially monodisperse nanobubbles and/or microbubbles formed by reversible transfer between the extruders and through the channel and porous, e.g., microporous or nanoporous, membrane can have a membrane, such as a lipid membrane, that defines at least one internal void, which includes the at least one gas. The lipid membrane can exhibit selective acoustic response to known ultrasound pressures. In some embodiments, the lipid membrane can include, for example, the plurality of lipids, the edge-activator, which is incorporated between lipids of the membrane and enhances the flexibility of the nanobubbles, the membrane stiffener, which is incorporated on an outer surface of the membrane and enhances the membranes resistance to tearing, and, other additives, such as pluronic (poloxamer), alcohols and cholesterols, that change the modulus and/or interfacial tension of the bubble shell.

In other embodiments, each of the nanobubbles or microbubbles so formed can include a hydrophilic outer domain at least partially defined by hydrophilic heads of the lipid and a hydrophobic inner domain at least partially defined by hydrophobic tails of the lipid. An edge activator, such as propylene glycol, can at least partially extend between the lipids from the outer domain to the inner domain. The glycerol can be provided on the outer domain of the nanobubbles and extend partially between hydrophilic heads of the lipids. The gas, which is encapsulated by the membrane, can include, for example, a perfluorocarbon, such as perfluoropropane or perfluorobutane, sulfur hexafluoride, carbon dioxide, nitrogen (N₂), oxygen (O₂), and air.

The membranes defining the nanobubbles can be concentric or otherwise and have a unilamellar configuration (i.e., comprised of one monolayer or bilayer), an oligolamellar configuration (i.e., comprised of about two or about three monolayers or bilayers), or a multilamellar configuration (i.e., comprised of more than about three monolayers or bilayers). The membrane can be substantially solid (uniform), porous, or semi-porous.

In some embodiments, the nanobubble can have a size that facilitates extravasation of the nanobubble in cancer therapy or diagnosis. For example, the nanobubble can have a size (diameter) of about 30 nm to about 600 nm or about 100 nm to about 500 nm (e.g., about 300 nm), depending upon the particular lipids, edge activator, and membrane stiffener as well as the method used to form the nanobubble. In other embodiments, the nanobubbles can have a mean diameter of about 0.5 μm to about 0.4 μm, preferably, about 0.1 μm to about 0.3 μm or about 0.1 μm to about 0.2 μm.

Optionally, following generation of the dispersion of the substantially monodisperse nanobubbles and/or microbubbles, at step 50 the dispersion of nanobubbles and/or microbubbles can be further processed by, for example, centrifugation, filtration, sonication, homogenization to remove non-buoyant particles and/or foam in the nanobubble and/or microbubble dispersions. Example of additional processing techniques, as well as others, are discussed, for example, in U.S. Pat. No. 4,728,578; U.K. Patent Application GB 2193095 A; U.S. Pat. Nos. 4,728,575; 4,737,323; International Application PCT/US85/01161; Mayer et al., Biochimica et Biophysica Acta, Vol. 858, pp. 161-168 (1986); Hope et al., Biochimica et Biophysica Acta, Vol. 812, pp. 55-65 (1985); U.S. Pat. No. 4,533,254; Mayhew et al., Methods in Enzymology, Vol. 149, pp. 64-77 (1987); Mayhew et al., Biochimica et Biophysica Acta, Vol 755, pp. 169-74 (1984); Cheng et al, Investigative Radiology, Vol. 22, pp. 47-55 (1987); PCT/US89/05040, U.S. Pat. Nos. 4,162,282; 4,310,505; 4,921,706; and Liposome Technology, Gregoriadis, G., ed., Vol. I, pp. 29-31, 51-67 and 79-108 (CRC Press Inc., Boca Raton, Fla. 1984). The disclosures of each of the foregoing patents, publications and patent applications are incorporated by reference herein, in their entirety.

In some embodiment, the dispersion of nanobubbles and/or microbubbles can be passed through a porous, e.g., microporous or nanoporous, filter. Filter pore sizes are selected for sizing as well as to remove any potential contaminants. The filter pore size may be between 10 nm and 1 μm, more preferably between 30 nm and 1 μm, and even more preferably between 100 nm and 1 μm, for example, about 0.45 μm.

Two or more filters may be stacked in a series to maximize the effectiveness of filtration. Useful materials for formation of the filters include hydrophilic polymers, such as polyether sulfone, polysulfonate, polycarbonate, and polyvinylidene chloride. In addition, glass, ceramics, and metal filters may also be utilized. Additionally, wire, polymer, or ceramic meshes may also be utilized. Filtration may be performed as part of the manufacturing process or during administration through an in-line filter.

For storage prior to use, the nanobubbles and/or microbubbles may be suspended in an aqueous solution, such as a saline solution (for example, a phosphate-buffered saline solution), or simply water, and stored preferably at a temperature of between about 2° C. and about 10° C., preferably at about 4° C. Preferably, the water is sterile. More preferably, the nanobubbles and/or microbubbles are stored in an isotonic saline solution, although, if desired, the saline solution may be a hypotonic saline solution (e.g., about 0.3 to about 0.5% NaCl). The solution also may be buffered, if desired, to provide a pH range of about pH 5 to about pH 7.4. Suitable buffers for use in the storage media include, but are not limited to, acetate, citrate, phosphate and bicarbonate.

Bacteriostatic agents may also be included with the nanobubbles and/or microbubbles to prevent bacterial degradation on storage. Suitable bacteriostatic agents include but are not limited to benzalkonium chloride, benzethonium chloride, benzoic acid, benzyl alcohol, butylparaben, cetylpyridinium chloride, chlorobutanol, chlorocresol, methylparaben, phenol, potassium benzoate, potassium sorbate, sodium benzoate and sorbic acid. One or more antioxidants may further be included with the gaseous precursor-filled liposomes to prevent oxidation of the lipid. Suitable antioxidants include tocopherol, ascorbic acid and ascorbyl palmitate.

Advantageously, the nanobubbles and/or microbubbles formed by the extrusion method described herein are substantially monodisperse and can have a smaller size compared to nanobubbles formed by mechanical agitation using similar lipid solutions and gases. The nanobubbles and/or microbubbles formed by the extrusion method described herein also have a more focused acoustic response compared to nanobubbles formed by mechanical agitation, which is advantageous for ultrasound targeted drug delivery applications.

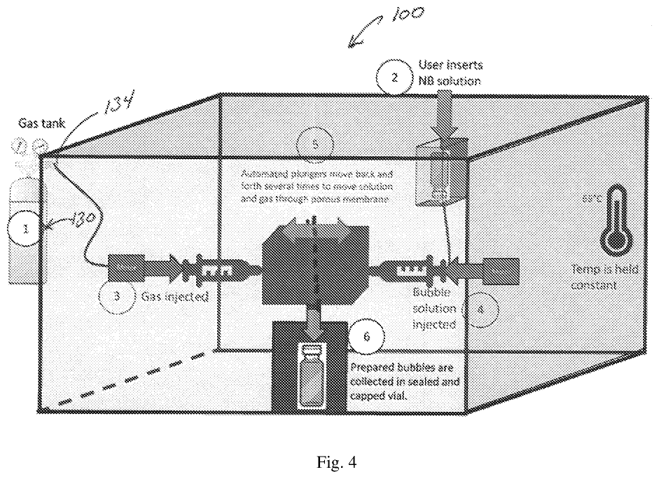

It will be appreciated that the method used to generate the plurality of gas-core, lipid, protein and/or polymer shelled nanobubbles and/or microbubbles can be performed using an apparatus that integrates the various components described herein. For example, FIGS. 3 and 4 are a schematic illustrations of an apparatus 100 for generating a plurality of gas-core, lipid, protein and/or polymer shelled nanobubbles and/or microbubbles using an extrusion process described herein. The apparatus 100 includes a housing 102 that includes a first extruder 110, a second extruder 112, a channel 114 fluidly connecting the first extruder 110 and second extruder 112, and a porous, e.g., microporous or nanoporous, membrane 116 provided in the channel 114.

The first extruder 110 is configured to receive a dispersion of at least one lipid solution and gas from a dispersion source 120 and transfer or inject the contents of the first extruder 110 through the channel 114 and porous, e.g., microporous or nanoporous, membrane 116 to the second extruder 112 by actuation of a plunger 122 of the first extruder 110. The dispersion source 120 can include a vial that contains the dispersion of lipid solution and gas that can be inserted in a first inlet port 124 of the apparatus 100. The first inlet port 124 is fluidly connected to the first extruder 110 via a first fluid interconnect 126.

The second extruder 112 is configured to receive gas from gas source 130 and transfer or inject the contents of the second extruder 112 through the channel 114 and porous, e.g., microporous or nanoporous, membrane 116 to the first extruder 110 by actuation of a plunger 132 of the second extruder 112. The gas source 130 can include a removable gas cartridge (FIG. 3) that can be inserted in a second inlet port 134 of the apparatus. Alternatively, as illustrated in FIG. 4, the gas source 130 can include a gas tank that is connected to the second inlet port 134. The second inlet port 134 is fluidly connected to the second extruder 112 via a second fluid interconnect 136.

The channel 114 is configured to permit reversible fluid flow to and from the first extruder 110 and the second extruder 112 and through the porous, e.g., microporous or nanoporous, membrane 114 provided in the channel 112. The first extruder 110 and the second extruder 112 are further configured to reversibly transfer the dispersion in first extruder 110 and the gas in second extruder 112 to and from the first extruder 110 and second extruder 112 through the channel 114 and porous, e.g., microporous or nanoporous, membrane 116 to provide a dispersion of lipid, protein and/or polymer shelled nanobubbles and/or microbubbles.

The channel 114 includes an outlet port 140 configured to transfer the dispersion of lipid, protein and/or polymer shelled nanobubbles and/or microbubbles from the channel 114 and housing 102 to a collection vessel 142, such as a collection vial, where the nanobubbles and/or microbubble can be sealed in the vessel. Optionally, the outlet port 140 can include a porous, e.g., microporous or nanoporous, filter (not shown) configured to filter the dispersion of lipid, protein and/or polymer shelled nanobubbles and/or microbubbles after reversibly transferring the dispersion of lipid, protein and/or polymer shelled nanobubbles and/or microbubbles. The porous, e.g., microporous or nanoporous, filter can have average pore diameter similar to or less than the average pore diameter than the porous, e.g., microporous or nanoporous, membrane.

The apparatus 100 further includes a first motor 150 contained within the housing 102 configured to actuate the first extruder 110 and a second motor contained within the housing 102 configured to the actuate the second extruder 112 such that the dispersion and gas are reversibly transferred though the porous, e.g., microporous or nanoporous, membrane 116 in the channel 114. The first motor 150 and the second motor 152 can actuate respectively the first extruder 110 and the second extruder 112 by moving the plungers 122,132 of the first extruder 110 and second extruder 112 back and forth to allow transfer of the dispersion and gas between the first extruder 110 and second extruder 112 through the channel 114 and porous, e.g., microporous or nanoporous, membrane 116.

The apparatus 100 further includes a heat source (not shown) contained within the housing 102 for maintaining the dispersion at a temperature where the at least one lipid is in a fluid state during reversible transfer of the dispersion and gas from and to the first extruder 110 and the second extruder 112. The heat source can be configured to maintain the temperature higher than the solid to fluid phase transition temperature of the at least one lipid, for example, about 37° C. to about 80° C., about 50° C. to about 75° C., or about 65° C.

In operation, a dispersion of lipid solution and gas provided in a vial 129 that is connected to the first inlet port 124 can be transferred to the first extruder, and gas from the gas source 130 that is connected to the second inlet port 134 can be transferred to the second extruder 112. Actuation of the first extruder 110 and the second extruder 112 by the first and the second motors 150,152 allows the dispersion and gas to mix and be reversibly transferred between the first extruder 110 and second extruder 112 through the channel 114 and porous, e.g., microporous or nanoporous, membrane 116 generating a dispersion of nanobubbles and/or microbubbles. The dispersion of nanobubbles and/or microbubbles is then transferred through the outlet port 140 to the collection vessel 142 for storage and use in diagnostic, therapeutic, and/or theranostic applications.

In some embodiments, the monodisperse nanobubbles and/or microbubbles generated using the method, system, and/or apparatus described herein can be administered to a subject for diagnostic, therapeutic, and/or theranostic applications. For example, monodisperse nanobubbles formed by the method described herein can be administered to a subject for imaging at least one region of interest (ROI) of the subject. The ROI can include a particular area or portion of the subject and, in some instances, two or more areas or portions throughout the entire subject. The ROI can include, for example, pulmonary regions, gastrointestinal regions, cardiovascular regions (including myocardial tissue), renal regions, as well as other bodily regions, tissues, lymphocytes, receptors, organs and the like, including the vasculature and circulatory system, and as well as diseased tissue, including neoplastic or cancerous tissue. The ROI can include regions to be imaged for both diagnostic and therapeutic purposes. The ROI is typically internal; however, it will be appreciated that the ROI may additionally or alternatively be external.

In some embodiments, the nanobubbles used to image the ROI can be formulated such that the internal void of at least one of the nanobubbles includes at least one contrast agent. For example, a contrast agent (in either liquid or gaseous form) can be contacted with the hydrated lipid/propylene glycol/glycerol solution under conditions effective to entrap the contrast agent in the internal void of the nanobubble. For instance, sealed vials containing a lipid (DBPC/DPPA/DPPE/mPEG-DSPE)/propylene glycol/glycerol solution can have the air withdrawn by a syringe and then octafluoropropane added until the pressure in the vial is equalized. Other examples of contrast agents (besides octafluoropropane) that may be incorporated into the nanobubbles are known in the art and can include stable free radicals, such as, stable nitroxides, as well as compounds comprising transition, lanthanide and actinide elements, which may, if desired, be in the form of a salt or may be covalently or non-covalently bound to complexing agents, including lipophilic derivatives thereof, or to proteinaceous macromolecules.

Since nanobubble size may influence biodistribution, the size of the nanobubbles can be selected depending upon the region of interest (ROI) of the subject. For a ROI comprising an organ (e.g., a liver or kidney) the size of the nanobubbles may be larger than for a ROI comprising tumor tissue. Where the ROI comprises, for example, tumor tissue and differentiation between the tumor tissue and normal or healthy tissue is sought, smaller nanobubbles may be needed to penetrate the smaller venuoles and capillaries comprising the tumor tissue. It should be appreciated that the nanobubbles can comprise additional constituents, such as targeting ligands to facilitate homing of the nanobubbles to the ROI.

The nanobubble composition can be administered to the subject via any known route, such as via an intravenous injection. By way of example, a composition comprising a plurality of octafluoropropane-containing nanobubbles can be intravenously administered to a subject that is known to or suspected of having a tumor.

At least one image of the ROI can be generated using an imaging modality. The imaging modality can include one or combination of known imaging techniques capable of visualizing the nanobubbles. Examples of imaging modalities can include ultrasound (US), magnetic resonance imaging (MRI), nuclear magnetic resonance (NMR), computed topography (CT), electron spin resonance (ESR), nuclear medical imaging, optical imaging, and positron emission topography (PET). The imaging modality can then be operated to generate a visible image of the ROI. In a subject known to or suspected of having a tumor, for example, an ultrasonic transducer can be applied to at least a portion of the ROI to image the target tissue. A visible image of the tumor can then be obtained, such that the presence, absence, and/or extent of a particular neoplastic disorder can be ascertained. It will be appreciated that the imaging modality may be used to generate a baseline image prior to administration of the composition. In this case, the baseline and post-administration images can be compared to ascertain the presence, absence, and/or extent of a particular disease or condition.

In other embodiments, the nanobubbles can be administered to a subject to treat and/or image a neoplastic disease in subject. Neoplastic diseases treatable by the present invention can include disease states in which there are cells and/or tissues which proliferate abnormally. One example of a neoplastic disease is a tumor. The tumor can include a solid tumor, such as a solid carcinoma, sarcoma or lymphoma, and/or an aggregate of neoplastic cells. The tumor may be malignant or benign, and can include both cancerous and pre-cancerous cells.

The location(s) where the nanobubble composition is administered to the subject may be determined based on the subject's individual need, such as the location of the neoplastic cells (e.g., the position of a tumor, the size of a tumor, and the location of a tumor on or near a particular organ). For example, the composition may be injected intravenously into the subject. It will be appreciated that other routes of injection may be used including, for example, intramuscular, intraarterial, intrathecal, intracapsular, intraorbital, intracardiac, intradermal, intraperitoneal, transtracheal, subcutaneous, subcuticular, intraarticular, subcapsular, subarachnoid, intraspinal and intrasternal routes.

Since nanobubble size may influence biodistribution, the size of the nanobubbles can be selected depending on the neoplastic disorder being imaged and/or treated. Where the neoplastic disorder comprises tumor tissue, smaller nanobubbles may be needed to penetrate the smaller venuoles and capillaries comprising the tumor tissue.

In some embodiments, ultrasound can be used as a remote source to provide locoregional destruction or fragmentation of the nanobubbles to release a therapeutic agent (e.g., chemotherapeutic agent) provided in the nanobubbles to the tissue or region of interest (e.g., cancer or tumor). Advantageously, ultrasound can release the chemotherapeutic agent from the nanobubbles and enhance the anti-tumor efficacy chemotherapeutic agent by ultrasonic cavitation effects, sound effect and other effects. The radiation ultrasound also improves the cell membrane permeability of the chemotherapeutic resulting in much more chemotherapeutic agent in the tumor cells.

It will be appreciated that other remote sources can be used to provide locoregional destruction or fragmentation of the nanobubbles to release the therapeutic agent. These other remote sources can include, for example, high intensity focused ultrasound (HIFU) and radiofrequency ablation.

It will be appreciated that the monodisperse nanobubbles described herein can be used in other applications besides diagnostic, therapeutic, and theranostic applications described above. Nanobubble ultrasound contrast agents have shown great potential in areas of health including cardiovascular and eye diseases, as well as neuromuscular disorders such as Duschenne Muscular dystrophy. Inflammation has been associated with hypoxia. Nanobubbles can deliver oxygen to hypoxic cell and tissues and can be a potential treatment option.

Although the use of nanobubbles in medicine is in an early development stage, it is possible that in the future, the applications of nanobubbles in medicine will be as far reaching if not more than that of microbubbles whose applications span across the areas of malignant, infectious, cardiovascular and autoimmune diseases.

The following Example is for the purpose of illustration only and is not intended to limit the scope of the claims, which are appended hereto.

### Example

In this example, we describe an alternative highly efficient technique for nanobubble formulation using a commercially available mini-extruder setup. The principle behind this system is to pass a dispersion of lipids (one or a mixture) several times through a porous membrane to arrive at a dispersion of monodispersed vesicles/liposomes. The formation of the vesicles/liposomes is made possible by extrusion if the processes take place at a temperature higher than their phase transition temperature such that the lipids are in a fluid state. While commonly utilized as a strategy for formulating liposomes, to our knowledge, the work presented here is the first to demonstrate the ability to form stable monodisperse gas-core nanostructures using the mini-extruder system.

This example alsopresents a comparison of NBs obtained using two different methods: (a) using mechanical agitation (Vialmix) to generate the bubbles followed by differential centriguation (v-NBs) and (b) using extrusion to form NBs and centrifugation to remove the foam (e-NBs). The NBs were characterized and compared with regard to size, concentration, yield, and non-linear acoustic response. Our results show that the extrusion method produces a yield that is at least 5 times higher in volume, with a similar quantity of bubbles when 49% less PL is used initially compared to the control method. The extrusion method presented here also produced smaller (≈160 nm vs≈320 nm) and more monodisperse (full width at half maximum, FWHM, of 190 nm vs 113 nm) NBs without drastically impacting the acoustical response of the agents. This example further describes three parameters that can be modified and optimized on the extruder process. The three parameters that can be modified and optimized include: (a) the temperature of extrusion, (b) the concentration of the lipid solution and (c) the number of passes through the extruder. The effect of these parameters were studied to show how each can interfere and influence NB formation by the extrusion process. Finally, we present an in vivo experiment with e-NBs lightly modified in terms of size isolation. The final e-NBs provided similar initial response as v-NBs and a stable signal up to 10 min.

### Experimental Section/Methods

### Raw Materials

Phospholipids (PL) including DBPC (1,2-dibehenoyl-sn-glycero-3-phosphocholine), DPPA (1,2 dipalmitoyl-sn-glycero-3-phosphate), and DPPE (1,2-dipalmitoyl-sn-glycero-3-phosphoethanolamine) were obtained from Avanti Polar Lipids (Pelham, AL), and mPEG-DSPE (1,2-distearoyl-sn-glycero-3-phosphoethanolamine-N-[methoxy(polyethylene glycol)-2000](ammonium salt)) was obtained from Laysan Lipids (Arab,AL). Propylene glycol (PG) was purchased from Sigma. Glycerol was purchased from Acros Organics (Morris, NJ). Octafluoropropane was obtained from AirGas (Cleveland, OH). Sterile PES syringe filter, 0.45 μm pore size, 30 mm and sterile MCE syringe filter, 0.8 μm pore size, 33 mm, were purchased from Celltreat and Millipore respectively.

Fabrication of Mechanical Agitation Nanobubbles (v-NBs)

Mechanical agitation formulation of lipids shelled v-NBs stabilized with octafluoropropane (C₃F₈) has been described previously. Briefly, lipids including DBPC (60.1 mg), DPPA (10 mg), DPPE (20 mg), and mPEG-DSPE (10 mg) were dissolved in propylene glycol (PG) (1 mL). 9 mL of a mixture of glycerol (Gly) and phosphate buffer saline (PBS) (1:8, v:v) was added to the lipid solution after dissolution of the PL at 80° C. In a sealed 3 mL vial, the lipid solution (1 mL) was added and the air inside was replaced with C₃F₈. Finally, the vial was placed on a VialMix shaker (Bristol-Myers Squibb Medical Imaging, Inc., N. Billerica, MA) for 45 s to drive bubble self-assembly. v-NBs were isolated from the mixture by centrifugation at 50 rcf for 5 min with the vial inverted. 500 μL v-NBs were obtained from the vial.

Fabrication of Extruder Nanobubbles (e-NBs)

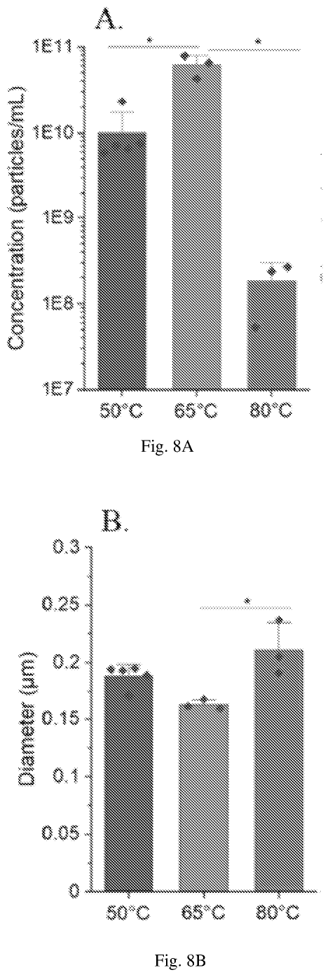

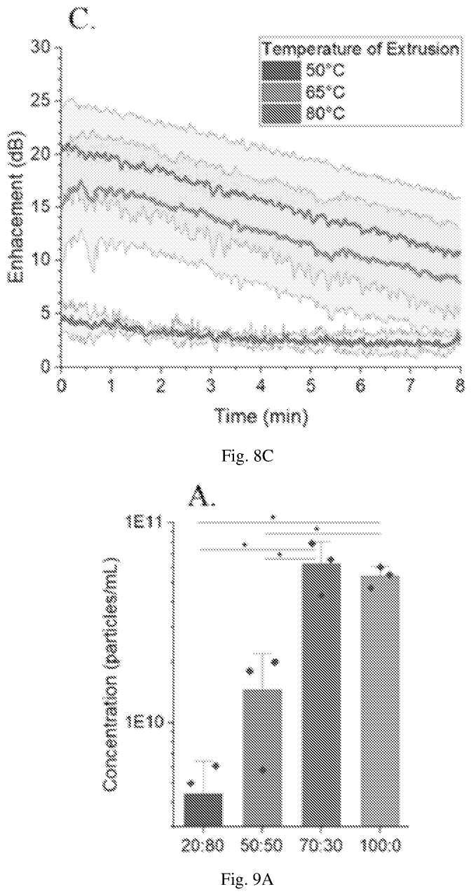

Extruder formulation of lipid shelled e-NBs stabilized with octafluoropropane (C₃F₈) were made from the same lipid solution mixture as mechanical agitation formulation v-NBs previously described. Briefly, lipids including 60.1 mg of DBPC, 10 mg of DPPA, 20 mg of DPPE, and 10 mg of mPEG-DSPE were dissolved in 1 mL of propylene glycol (PG). 9 mL of a mixture of glycerol and phosphate buffer saline (PBS) (1:8, v:v) was added to the lipid solution after dissolution of the PL at 80° C. This lipid solution (7 mL) was then diluted with PG:Gly:PBS (1:1:8 v:v:v) mixture (3 mL). In a sealed 3 mL vial, the dilute lipid solution (1 mL) was added and the air inside was replaced with C₃F₈. A mini-extruder from Avanti Polar Lipid, well known for the formulation of liposomes/vesicles, was used to make the e-NBs. The extruder was set-up with a 0.8 μm pore diameter polycarbonate membrane. Lipid solutions of 2 mg mL⁻¹, 5 mg mL⁻¹, 7 mg mL⁻¹ (e-NBs reference concentration) and 10 mg mL⁻¹ (v-NBs reference concentration) were tested. Before starting the extrusion process, the system (syringe and extruder) was equilibrated to the appropriate temperature (50° C.-80° C.) for 10 min. The solution was passed through the extruder 10-40 times. At the end of the extrusion process, the empty syringe was removed and the solution was passed for the last time through the extruder for collection in a 15 mL falcon tube. Both syringes were then washed with PG:Gly:PBS. Centrifugation at 30 rcf for 2 min was used to quickly discard the foam formed during the extrusion process. The liquid part was then passed through a 0.45 μm PES membrane filter. Between 2.5 and 3 mL of e-NBs solution was obtained after filtration (FIG. 8).

For in vivo measurement, the e-NBs production protocol was modified. Here, after the extrusion and washing process, the formulation was directly passed to a sterile MCE syringe filer of 0.8 μm pore size without centrifugation. This modification allowed us to prepare NBs which gave significant contrast under in vivo conditions.

### Characterization of NB Morphology, Size, and Concentration

### Resonant Mass Measurement (RMM)

The size distribution, concentration, and buoyant mass of NBs were measured using resonant mass measurement (RMM) (Archimedes, Malvern Pananalytical Inc., Westborough, MA, USA) using a calibrated nanosensor (100 nm-2 μm). Sensors were pre-calibrated using NIST traceable 565 nm polystyrene bead standards (ThermoFisher 4010S, Waltham MA, USA). E-NBs and v-NBs were diluted 1:100 and 1:1000 respectively with phosphate-buffered saline (PBS, pH 7.4) before measurement. A total of 500 particles were measured for each trial (n >3). The SD is the one of the trials only.

### Transmission Electron Microscopy (TEM)

Bubble morphology was imaged with a transmission electron microscope (TEM; Tecnai™ G2 Spirit BioTWIN, FEI Company) operated at 120 kV based on a previously reported method (Owen and Stride, 2015). 10 μL of a dilute suspension of v-NBs and e-NBs, at 1:100 and 1:10 dilution respectively, were placed in an inverted position for 2 min on a 400 mesh Formvar®-coated copper grid. The sample was then stained by placing it on top of a 20 μL droplet of 2% uranyl acetate for 30 s and the excess was removed. The TEM grid containing the bubble sample was allowed to dry for another 30 min.

### Stability Under Ultrasound

Stability under ultrasound was studied in a “T” agarose phantom (FIG. 3D) with an ultrasound transducer (PLT-1204BT) placed directly on the top, in contact with the phantom and the media. The phantom was filled with a total of 20 mL of certain volume of e-NBs or v-NBs dispersion in PBS. For the controlled e-NBs and v-NBs comparison, NBs were diluted in PBS to satisfy a matched 0.02 μL gas volume. However, for the evaluation of the extruder parameters the dilution of e-NBS in PBS was fixed at 1:100 dilution. In all cases, the solution was stirred at 700 rpm through the full duration of imaging acquisition. Before starting the acquisition, the solution was stirred at 1000 rpm for 10 s to reach an approximately homogenous distribution of NBs throughout the imaging plane. Nonlinear contrast images were continuously acquired using a clinical US scanner (AplioXG SSA-790A, Toshiba Medical Imaging Systems, Otawara-Shi, Japan) via contrast harmonic imaging (CHI, 12 MHz, mechanical index 0.22, focus depth of 0.75 cm, 2D gain of 70 dB, dynamic range of 65 dB) at 1 frame per second for 8 min. Raw echo power data was recorded and analyzed using a built-in CHI-Q software. Images were analyzed using quantification software (CHI-Q) available on the scanner. Using the software, the mean intensity of the backscattered nonlinear ultrasound signal over time was measured in selected regions of interest, and these values were used to create the time intensity curves (TIC). For the in vitro data to quantify the extent of pressure dependent NB activity, the signal was measured in 3 zones (Z1 above the focus, Z2 at the focus, and Z3 below the focus). For each zone, the background signal was subtracted prior to constructing the TIC. Signal decay over time was determined from this data (FIG. 4.). Experiments were repeated in triplicate.

### In Vivo Ultrasound Imaging

Mice were handled according to a protocol approved by the Institutional Animal Care and Use Committee (IACUC) at Case Western Reserve University and were in accordance with all applicable protocols and guidelines in regards to animal use. Male athymic nude mice (4-6 weeks old) were anesthetized with inhalation of 2% isoflurane (100 mL min⁻¹ air). Tail vein administration of 200 μL of undiluted in vivo e-NBs (0.8 μm filter e-NBs described previously), or diluted to the same gas volume v-NBs, were performed. The same US probe as above was placed to visualize kidney and liver. Contrast harmonic imaging (CHI, frequency, 12.0 MHz; MI, 0.2; dynamic-range, 65 dB; gain, 70 dB; imaging frame rate, 0.2 frames s⁻¹) was used to determine the change of tissue contrast during a 30 min imaging period. At least 3 flash replenish pulses (high energy pulses) were done between e-NBs and v-NBs measurements, to remove the remaining NBs. After 30 min of waiting time other type of bubble injected. Raw echo power data was recorded and was again analyzed using a built-in CHI-Q software as described above. Signal decay over time was determined from the data. The kidney and liver area was delineated by drawing regions of interest. The experiments were carried out in triplicate.

### Statistical Analysis

All experiments were carried out with minimum of triplicates, and the results were reported as mean±standard deviation (SD). One-way ANOVA with Tukey's multiple comparisons was carried out using Origin Lab 2020b to compare the means. Statistical significance was recorded as *p<0.05.

### Results and Discussion

Mechanical agitation (via Vialmix), sonication, and microfluidic devices are well-known techniques used to obtain nano and microbubbles. On the other hand, mini-extruders are commonly used to produce monodisperse vesicles and liposomes. This instrument consists of a polycarbonate membrane placed in between two filters sealed by two O-rings made of Teflon®. Passing a phospholipid solution several times through this membrane at a temperature higher than the transition temperature of the PL induces the formation of monodisperse vesicles. However, we found that the potential use of mini-extruders is not only limited to the formation of liquid core self-assembly but can also be extended to produce gas core vesicles such as NBs. The NBs obtained with this new technique have been characterized and compared to those commonly produced in our group using a mechanical agitation with regard to size, concentration, ultrasound response and signal decay rate.

Extruder vs. Mechanical Agitation

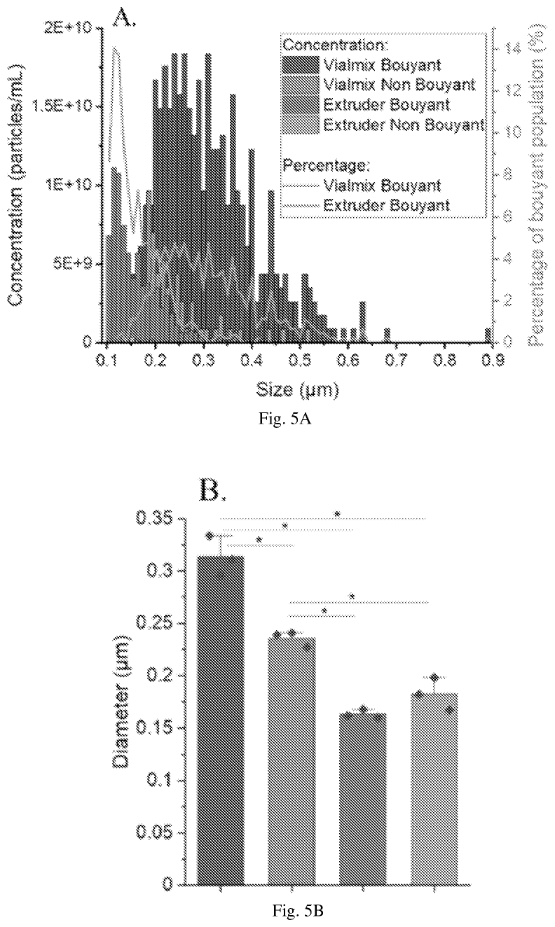

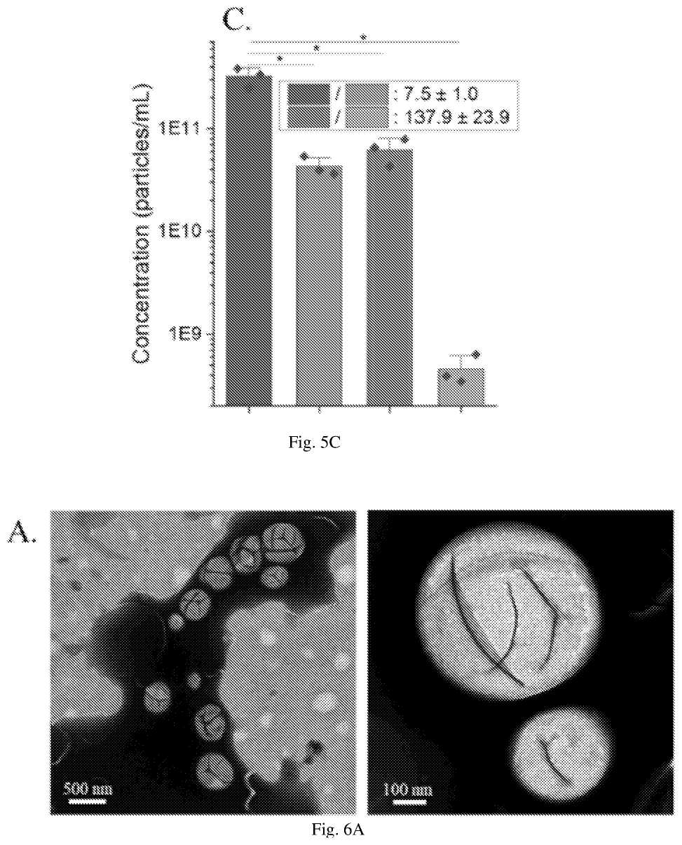

The mechanical agitation and extruder methods produced NB solutions with different characteristics (FIG. 5) as determined by resonant mass measurement (RMM).^{[41]} With the standard mechanical agitation via Vialmix (v-NB), 0.5 mL of NBs at a concentration of 3.2±0.7×10¹¹ v-NBs mL⁻¹ were obtained (FIGS. 5A and C), with a mean diameter of 0.32±0.1 μm for each trial according to the RMM with ±0.02 μm variation of the mean radius between the 3 measurements. (FIG. 5B). With the extruder (e-NB), a higher volume of solution was obtained, between 2.5 and 3 mL at a concentration at 6.2±1.8×10¹⁰ e-NBs mL⁻¹ (FIG. 5C). The mean diameter was reduced to 0.16±0.05 μm for each trial according to the RMM with a ±0.004 μm variation of the mean radius between the 3 measurements (FIG. 5B). The extruder povided more monodisperse and more reproducible NBs samples. The minimum volume obtained with the mini-extruder (2.5 mL) provided the same quantity of bubbles as mechanical agitation: 1.6×10¹¹ NBs using 49% less of the starting phospholipid mixture. Moreover, the mini-extruder produced e-NBs that were more monodisperse as demonstrated by a Full Width at Half Maximum (FWHM) of 0.113 μm vs 0.190 μm for v-NBs solution, and a smaller size range with e-NBs having bubble size distribution between 0.1 μm and 0.4 μm e-NBs in comparison to 0.2 μm to 1 μm for v-NBs (FIG. 5A). All the bins of v-NBs population represent less than 5% of the total concentration of the NBs. However for e-NBs all the bins between 0.1 and 0.2 μm had a percentage higher than 4%, and between 0.104 and 0.144 is even higher than 8% (FIG. 5A), further confirming that the mini-extruder produced more monodisperse agents.

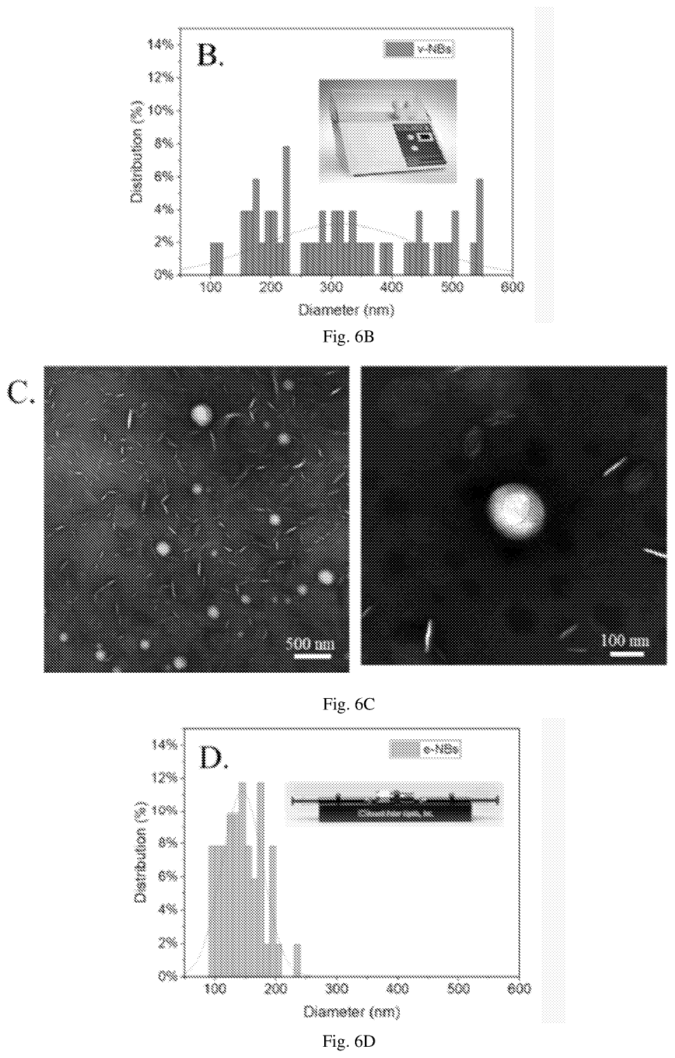

In addition, using the extruder for NB formulation produced proportionally fewer non-buoyant particles in the sample. The ratio for buoyant (bubbles) to non-buoyant particles was 8:1 for v-NBs compared to 138:1 for e-NBs (FIG. 6C). This result from e-NBs was unexpected considering the main use of the mini-extruder method is for the production of non-buoyant particles. These results show that the mini-extruder is a powerful technique for the formulation of a high yield monodisperse populations of NBs with a diameter lower than 200 nm.

The size of the NBs obtained with RMM was also compared to those obtained using Transmission electron microscopy (TEM) imaging analysis (FIG. 6). As expected, the mini-extruder generates significantly smaller NBs compared to the mechanical agitation method. Based on TEM, e-NBs have a diameter between 90 and 250 nm (FIG. 6B). With the lower limit of detection of the RMM, ˜100 nm, we can assume that the mean size and concentration measured are underestimated, since the smaller population of e-NBs formed will not be counted. In the case of v-NBs, the observed size range was from 100 to 550 nm (FIG. 6D). TEM results are in agreement with RMM with e-NBs having a smaller size range than v-NBs. Moreover, 9 of the 13 bins of the e-NBs histogram (FIG. 6D) represent 8% or more of the total population however only 1 under the 32 bins of the v-NBs histogram (FIG. 6B) corresponding to 8%. These results further confirm that the extruder method is a promising technique for the formation of monodisperse NBs.

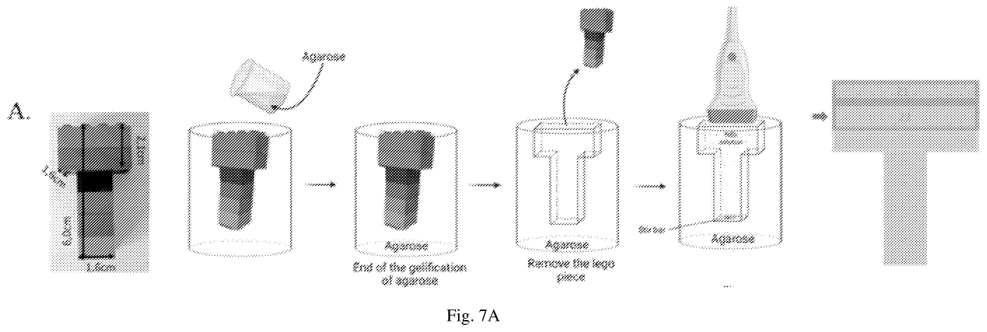

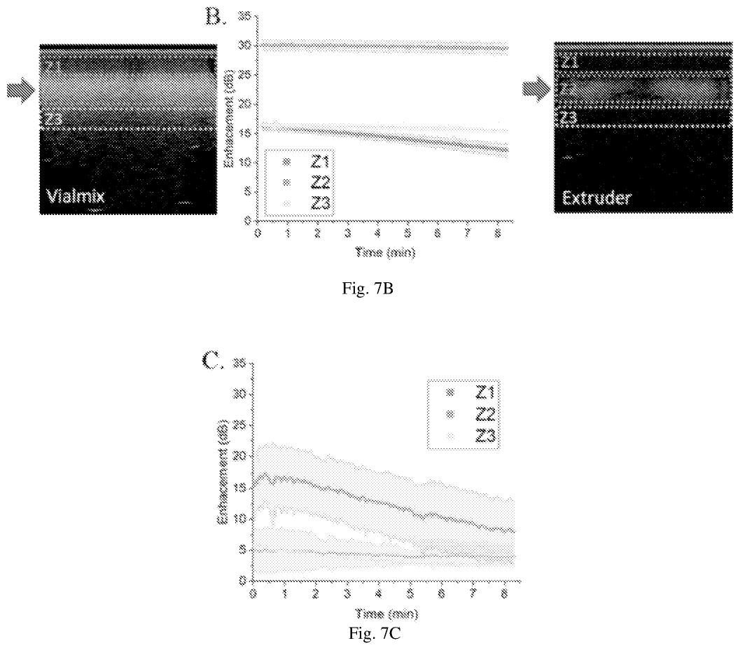

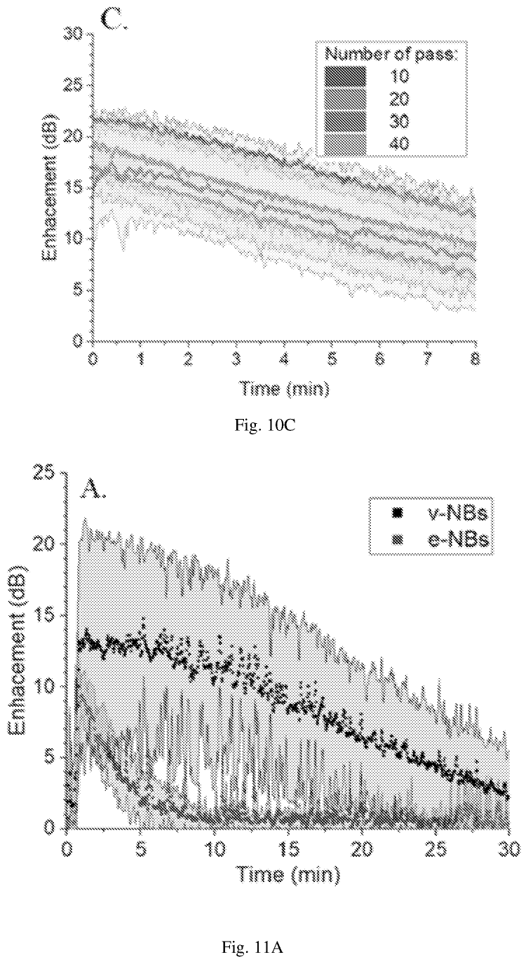

The acoustic response of NBs in aqueous solution (PBS) was evaluated in a custom-made “T”-shaped agarose hydrogel phantom with nonlinear contrast imaging mode using a commercial ultrasound scanner at 12 MHz (FIG. 7A). This setup allowed the transducer to be in direct contact with the bubble solution. The solution was agitated using a stir bar at the bottom of the phantom, out of the imaging field. e-NB and v-NB concentrations were normalized to the same theoretical gas volume calculated from the RMM measurements. The results show that v-NBs yield a higher initial acoustic response, (average of the triplicate and its SD) in the three regions of interest, Z1, Z2 and Z3, with 30±1 dB comapred to 17±5 dB for e-NBs for region Z2, the area at the focal point (FIGS. 7B and C). For e-NBs signal was primarily apparent in the area at the focal zone compared to v-NBs where significant activity was also seen above and below the focus. For e-NBs signal in Z2 represents 65±10% of total region of interest (ROI) compared to 48±1% for v-NBs (FIG. 10). This result suggests a strong pressure-dependent acoustic response of both NB types, which, as predicted, becomes more apparent with a more monodisperse bubble population. A smaller NB size predictably contributes to lower acoustic activity from the e-NBs at the same imaging pressures (mechanical index of 0.22), despite a comparable gas volume. In addition, the stability of the agent in the acoustic field is somewhat lower for the e-NBs. After 8 min of continuous data acquisition at 1 frame per second, minimal signal decay was observed for v-NBs. For e-NBs a 50% decay of the initial signal was observed from 17±5 dB to 8±5 dB (FIG. 7C). However, as described above, monodispersity of bubble populations may be desired to yield a uniform acoustic signal, which can be valuable for precise imaging and focused therapy with minimized off target effects. Despite the faster decay under ultrasound exposure, the acoustic response of e-NBs is considerably more localized, which can have a strong appeal for precision diagnostic and theranostic applications.

### Impact of Extruder Parameters

### Temperature

Temperature is a parameter which needs to be controlled during extrusion. We examined NB formulation at 50, 65 and 80° C. to determine the optimal parameter for formulation of buoyant particles and assessed the effect of temperature on e-NB size, yield and ultrasound signal intensity over 8 min. At 65° C. resulting e-NB had the smallest diameter, highest concentration and strong acoustic activity (FIG. 8). Further increasing the temperature to 80° C. resulted in a decreased bubble concentration (from 6.2±1.8×10¹⁰ at 65° C. to 1.4±1.3×10⁸ e-NBs.mL⁻¹ at 85° C., FIG. 8A) and a significant drop in acoustic activity in the focal zone from 17±5 dB at 65° C. to 4±1 dB at 80° C. (FIG. 8C). This data suggests that 80° C. is ideal to form nonbuoyant particles because it is higher than the transition temperature of the phospholipids used in the formulation. Lowering the temperature to 50° C. from 65° C., also resulted in a decrease in bubble concentration due to a reduction in the fluidity of the phospholipid solution, but an increase in bubble diameter. Reducing the fluidity of the phospholipid solution, in turn, reduces the efficiency of the formation of foam and consequently, the formation of the NBs. Interestingly, the acoustic response at 50° C. is comparable to the one performed at 65° C. with 21±4 dB and 17±5 dB, respectively (FIG. 8C). This suggests that bubbles produced at 50° C. to provide a significant acoustic response, potentially due to their larger diameter. For 50° C. and 80° C. the sizes of the NBs were 0.19±0.01 μm and 0.21±0.02 μm, respectively, which is higher compared to 65° C. at 0.16±0.004 μm (FIG. 8B). As with lipid nanoparticle formulation, temperature is a crucial factor in forming an efficient NBs. Temperature affects the fluidity of the solution, which is critical to efficiency pass the solution through the extruder without producing liquid-core self-assembly. A temperature higher than the transition phase of the PL, corresponding to 80° C. here, will result in the formation of more non buoyant particles. On the contrary, if the temperature is too low, here at 50° C., the lipids are not fluid enough to easily pass through the 0.8 μm membrane. 65° C. is the perfect intermediate temperature, which provided a solution fluid enough to pass through the extruder without reaching the transition phase of all PL.

### Lipid Concentration

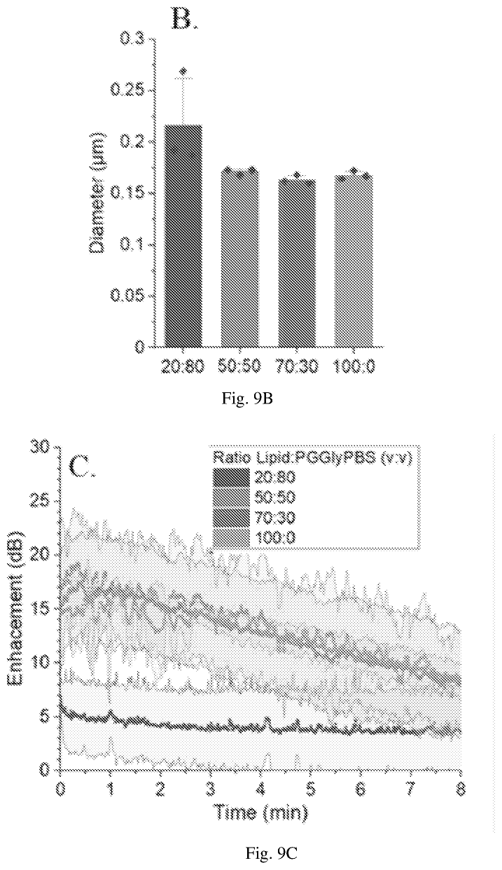

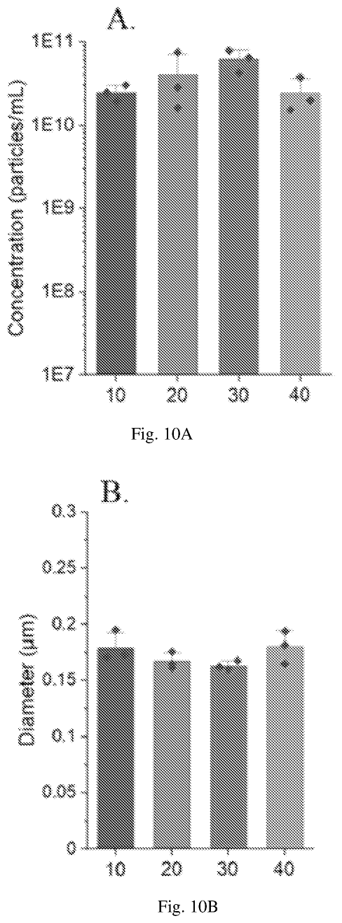

Using mechanical agitation followed by centrifugation and filtration to produce monodisperse NBs can result in significant starting material loss of (˜50%). Initially, a highly polydisperse bubble population is formed, and upon isolation of NBs, from the solution, a significant portion of the lipids is discarded. Further changes to the technique along with adjustments to the dilution can be implemented to help reduce material loss and improve process efficiency. Here we examined the effect phospholipid (PL) concentration on e-NB formulation. The standard v-NBs formulation consists of 10 mL solution at 10 mg mL⁻¹ of PL dilute in a mixture of PG, Gly, and PBS. In these experiments PL concentrations of 2 mg mL⁻¹ (20:80), 5 mg mL⁻¹ (50:50), 7 mg mL⁻¹ (70:30) and 10 mg mL⁻¹ (100:0), in the same mixture of solvent, were also investigated. Results show that decreasing the PL concentration has a significant impact on the NB concentration, size, and acoustic response (FIG. 9). Low PL concentrations result in low e-NB yield and an increase in NB size (from 0.21±0.05 μm for 2 mg mL⁻¹ to 0.160±0.004 μm for 7 mg mL⁻¹, FIG. 9B.). This observation is similar to previous reports using microfluidic bubbles wherein a decrease in the proportion of PL in the solution generated very unstable bubbles, which tend to coalesce more easily. At 5 mg mL⁻¹ the NB concentration was significantly lower (1.5±0.8×10¹⁰ e-NBs mL⁻¹) than at 7 mg mL⁻¹ (6.2±1.8×10¹⁰ e-NBs mL⁻¹) while a ratio of 10 mg mL⁻¹ was slightly lower (5.4±0.6×10¹⁰ e-NBs mL⁻¹). Despite these changes in concertation the ultrasound signal for each ratio was unchanged with the exception of the lowest PL concentration (17±5 dB vs 5±3 dB, respectively—FIG. 9C). Ultimately, we selected, the 7 mg mL⁻¹ ratio was used for the optimized protocol.

### Number of Extruder Passes

The number of passes through the extruder influences the polydispersity of resulting nanoparticles. An increase in the number of times the solution passes through the extruder typically leads to more monodisperse vesicles or liposomes. In our experiments, we examined a range of 10-40 passes. Surprisingly, no significant differences were observed in e-NB properties (FIG. 10). The concentration slightly increased comparing 10-pass to 30-pass samples from 2.5±0.5×10¹⁰ to 6.2±1.8×10¹⁰ e-NBs mL⁻¹ and then decreased for the 40-pass samples. This suggests that that the majority of e-NBs are formed in the first steps of the extrusion process. This was also confirmed by comparable acoustic response for the different number of passes used (FIG. 10C). In the case of the e-NBs, the monodispersity of the samples was improved by the removal of bigger bubbles with the combination of centrifugation and the use of a 0.45 μm PES membrane filter following extrusion. However, based on these results, 30 passes can be chosen as the optimal protocol used to produce bubbles with a smaller size, high yield, and a good acoustic response.

In vivo Assessment of e-NBs

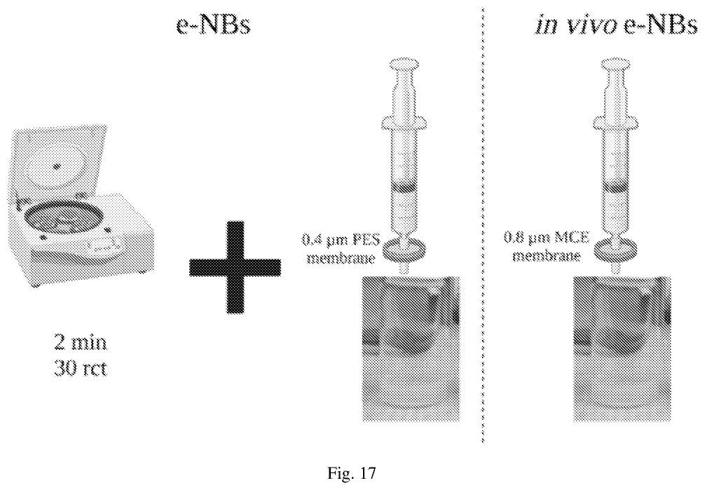

For the purpose of demonstrating in vivo activity, a variation of e-NB protocol was developed to achieve a significantly higher response from in vivo imaging. Here, after the extrusion, the resulting NB solution was directly passed through a 0.8 μm MCE filter and centrifugation was omitted (FIG. 17).

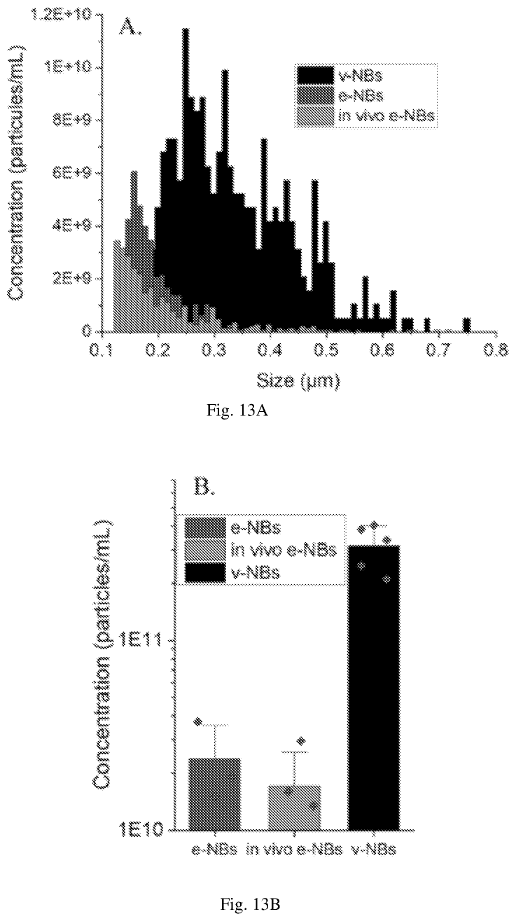

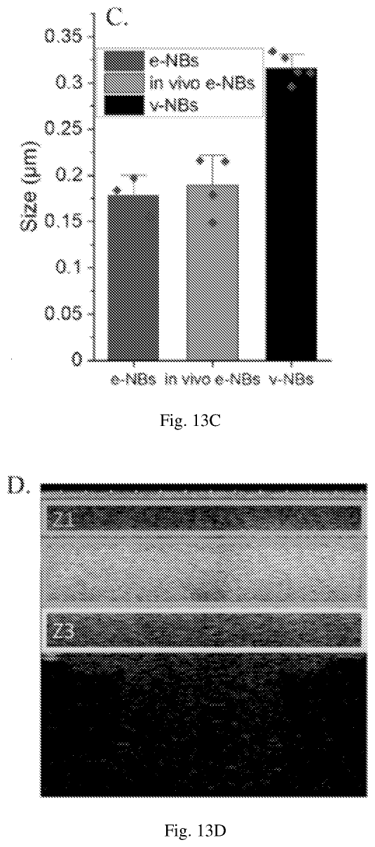

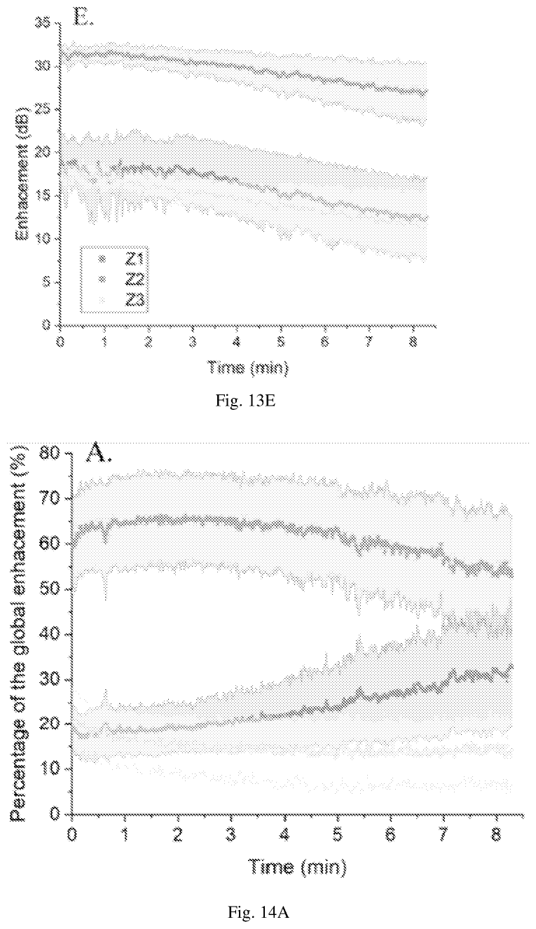

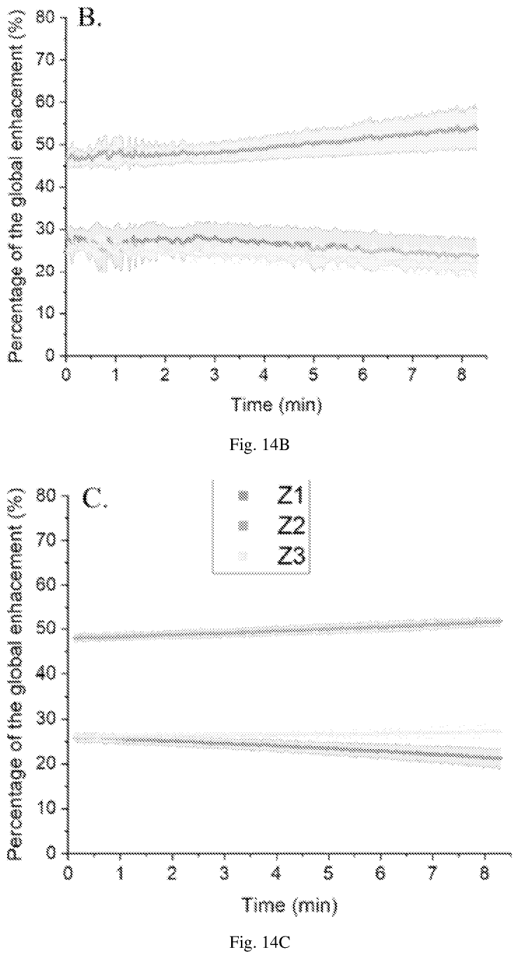

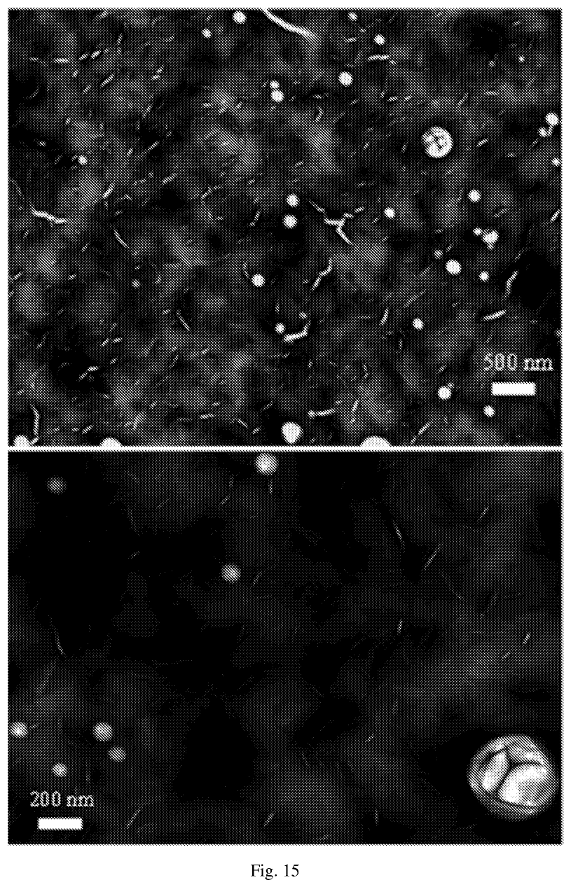

Because of their small size and monodispersity, e-NBs require higher acoustic pressures for generating non-linear activity (on which the CEUS sequences depend) which would not be practical for in vivo studies. As shown previously, a reduction in size on nanobubbles by a factor of 2 more than doubles the pressure threshold for non-linear activity. Therefore, when the mean size is shifted from 160 to 200 nm, the bubbles can be visualized in vivo with the same pressure and frequency as the v-NB (FIGS. 13 and 15).

The resulting in vivo e-NBs, had a size of ˜200 nm, with a similar distribution as the optimal e-NBs. Their in vitro acoustic response was also very similar to those of the v-NBs (FIG. 13). e-NBs were evaluated in healthy mice upon injection into the tail vein. Bubbles were normalized based on theoretical gas volume prior to injection.

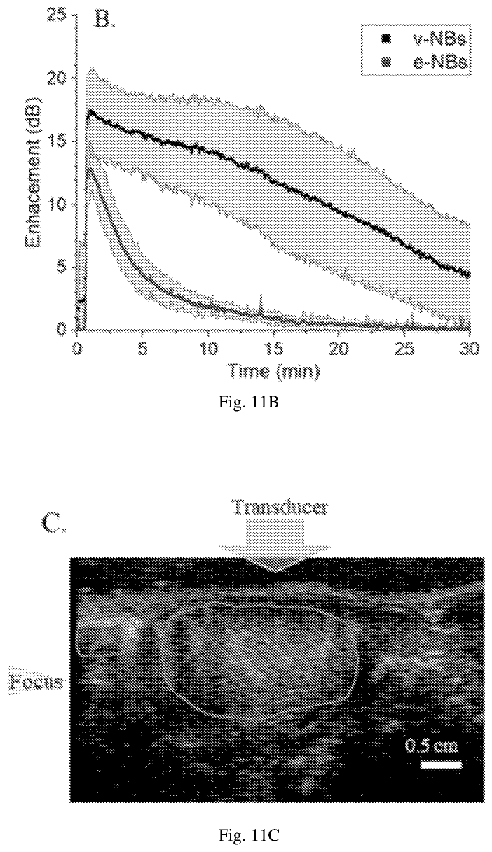

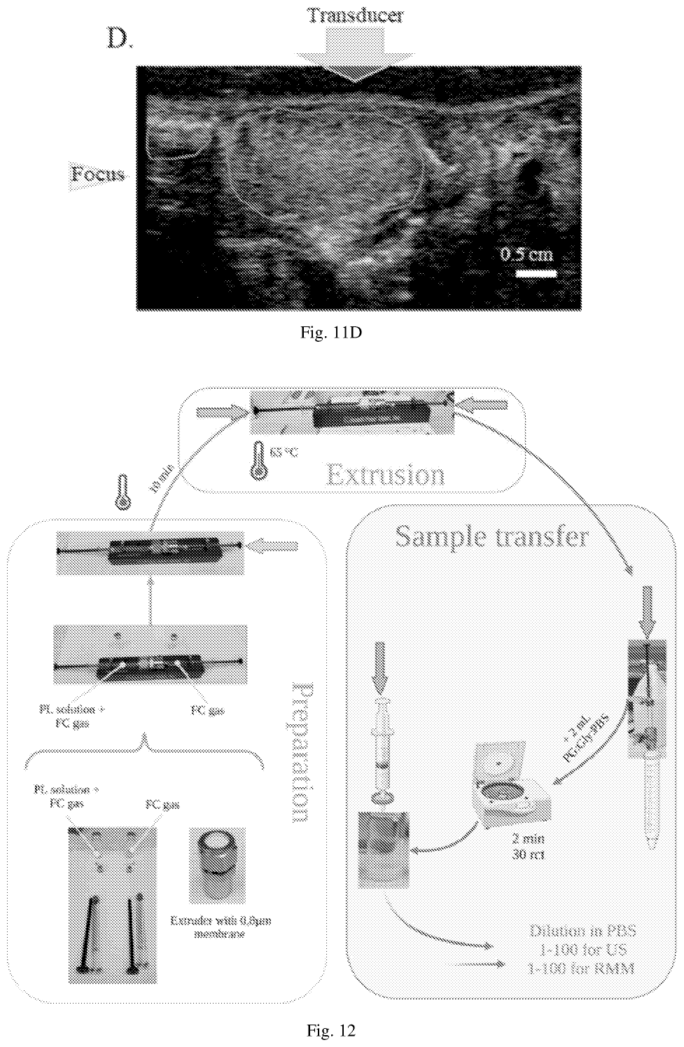

In vivo, the acoustic enhancement in the kidney (K) and the liver (L) of mice was measured for both v-NBs and e-NBs. The e-NBs had a lower initial peak response than v-NBs, with 9±2 dB compared to 13±8 dB respectively for the liver (FIG. 15A) and 13±2 dB compared to 17±4 dB respectively for the kidney (FIG. 11B). The response of in vivo e-NBs decreased at a higher rate than the v-NBs. This faster decay of in vivo e-NBs was likely a result of the smaller bubble size and improved monodispersity because of the sensitive dependence of acoustic response to NB diameter.

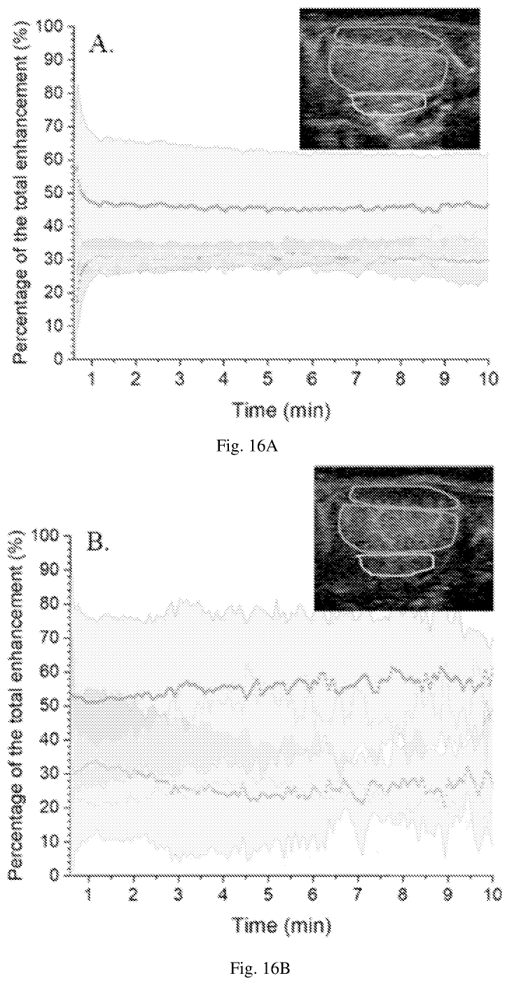

A comparison of the enhancement within the different imaging zones showed differences above and under the focus vs the focus area for the 2 bubbles categories. The focus region consistently yielded the highest enhancement. However, the average percentage of the focus zone compared to the global region of interest of the kidney from 30 s to 10 min is 56±16% for e-NBs compared to 46±12% for v-NBs (FIG. 16). This is consistent with the in vitro experiment. These results illustrate that, while attractive for various application, monodisperse bubbles may not be ideal for some in vivo imaging applications, where pressures can vary tremendously. Likewise, as discussed above, a faster decay of monodisperse nanobubbles was seen, which could also be driven by uniform decay in the acoustic field compared to the less-uniform decay of polydisperse formulations. Recent work demonstrated a unique microfluidic approach to formulate uniform NBs, by the formation of N₂/C₃F₈ MBs, which became NBs by dissolution of N₂ with time within the microfluidic reservoir. The obtained NB population using this method is of similar size scale as the e-NB, but a 100-1000 times lower concentration compared to the technique presented here (10⁷ vs 10¹⁰). The acoustic activity of the microfluidic NBs in vivo was shown for NBs that were greater than 300 nm in diameter. The same NBs were imaged in vivo for 20 seconds, with signal decay starting after 10 seconds. Importantly, the polydispersity can be tuned according to the ultimate application with simple modifications to the extrusion setup. Further specific modifications can be investigated in future applications to optimize extruded nanobubbles for diagnostic or therapeutic applications.

We have demonstrated, for the first time, the possibility of creating gas core self-assembly using a standard mini-extruder setup, which was previously used exclusively to form non-buoyant, liquid-core agents. The high yield e-NBs produced featured a smaller size and reduced polydispersity compared to NBs formed by mechanical agitation. We also demonstrated that the e-NBs have a more focused acoustic response, critical for ultrasound targeted drug delivery applications. The extrusion process is a complex mechanism where multiple parameters can impact the result of NBs formation. Temperature and lipid concentration are the two main factors that contribute to formation of small NBs with a high yield using the extrusion process. The temperature must be optimized to provide a good balance between the ability of the solution to cross the membrane without leading to the formation of non-buoyant vesicles, as determined by the fluidity of the solution. In terms of PL concentration, optimization can determine the minimum PL concentration needed to avoid the potential for bubble coalescence at low PL concentrations, and shear forces with increasing PL concentration. Overall, the extrusion technique presented here shows promise for simple, efficient and cost-effective NB production, with potential for straightforward scale up using existing strategies. Testing the extruded NBs in the biomedical imaging application showed feasibility of these particles as contrast agents with a strong and stable acoustic response, which is highly pressure dependent. The first in vivo study using extrusion produced NBs also showed significant signal enhancement in mouse kidney within the focal zone for an extended time. Additional optimization of the formulation and image acquisition parameters can yield a further improved imaging and therapeutic response.

From the above description of the invention, those skilled in the art will perceive improvements, changes and modifications. Such improvements, changes and modifications within the skill of the art are intended to be covered by the appended claims. All references, publications, and patents cited in the present application are herein incorporated by reference in their entirety.

1: A method of a generating a plurality of gas-core, lipid, protein and/or polymer shelled nanobubbles and/or microbubbles, the method comprising:
reversibly transferring a dispersion of at least one lipid, protein, and/or polymer and a gas through a porous membrane between a first depot and a second depot to provide a dispersion of lipid, protein and/or polymer shelled nanobubbles and/or microbubbles.

2: The method of claim 1, wherein the dispersion of at least one lipid, protein and/or polymer and gas is reversibly transferred through the membrane using a first extruder and a second extruder.

3: The method of claim 2, wherein the first extruder and the second extruder define, respectively, the first depot and the second depot.

4: The method of claim 1, wherein the reversible transferring of the dispersion through the porous membrane occurs at a temperature where the at least one lipid, protein and/or polymer is in a fluid state.

5: The method of claim 4, wherein the temperature is higher than the solid to fluid phase transition temperature of the at least one lipid.

6: The method of claim 1, wherein the first depot includes the dispersion of the at least one lipid, protein and/or polymer and gas and the second depot includes a gas prior to reversible transferring of the dispersion through the membrane.

7: The method of claim 1, wherein the dispersion is reversibly transferred through the porous membrane about 10 to about 80 times.

8: The method of claim 1, wherein the porous membrane has an average pore diameter of about 0.1 μm to about 2.0 μm.

9: The method of claim 1, further comprising optionally centrifuging the dispersion of lipid, protein and/or polymer shelled nanobubbles and/or microbubbles after reversibly transferring the dispersion of lipid, protein and/or polymer shelled nanobubbles and/or microbubbles through the porous membrane to remove any foam.

10: The method of claim 1, further comprising passing the dispersion of lipid, protein and/or polymer shelled nanobubbles and/or microbubbles through a porous filter after reversibly transferring or optionally centrifuging the dispersion of lipid, protein and/or polymer shelled nanobubbles and/or microbubbles.

11: The method of claim 10, wherein the porous filter average pore diameter similar to or less than the average pore diameter than the porous membrane.

12: The method of claim 1, wherein the dispersion of at least one lipid, protein and/or polymer and gas includes a lipid, protein and/or polymer solution, the lipid, protein and/or polymer solution having a lipid, protein and/or polymer concentration of about 1 mg/ml to about 20 mg/ml.

13: The method of claim 1, wherein the at least one lipid, protein and/or polymer includes a mixture of phospholipids having varying acyl chain lengths.

14: The method of claim 13, wherein the mixture of phospholipids includes at least two of dipalmitoylphosphatidylcholine (DPPC), dibehenoylglycerophosphocoline (DBPC), distearoylphosphatidylcholine (DSPC), diarachidonylphosphatidylcholine (DAPC), dioleoylphosphatidylethanolamine (DOPE), dipalmitoylphosphatidylethanolamine (DPPE), and distearoylphosphatidylethanolamine (DSPE); dipalmitoylphosphatidic acid (DPPA), or PEG functionalized lipids thereof.

15. (canceled)

16. (canceled)

17: The method of claim 12, wherein the lipid, protein and/or polymer solution includes further includes propylene glycol, glycerol, and phosphate buffered solution (PBS).

18: The method of claim 12, wherein the lipid, protein and/or polymer solution consists essentially of dibehenoylglycerophosphocoline (DBPC), dipalmitoylphosphatidic acid (DPPA), dipalmitoylphosphatidylethanolamine (DPPE), and PEG functionalized distearoylphosphatidylethanolamine (DSPE), propylene glycol, glycerol, and phosphate buffered solution (PBS).

19: The method of claim 1, wherein the gas of the dispersion of the at least one lipid, protein and/or polymer and gas includes a perfluorocarbon gas.

20. (canceled)

21: The method of claim 1, wherein the gas of the dispersion in the first depot is the same as the gas in the second depot.

22: The method of claim 1, wherein the nanobubbles have a mean diameter of about 0.05 μm to about 0.4 μm.

23: A system for performing the method recited in clam 1, the system comprising a first extruder configured to receive a dispersion of at least one lipid, protein and/or polymer and gas, a second extruder configured to receive at least one gas, a channel configured to permit reversible fluid flow to and from the first extruder and the second extruded, and a porous membrane provided in the channel, wherein the first extruder and the second extruder are further configured to reversibly transfer the dispersion in first extruder and the gas in second extruder to and from the first extruder and second extruder through the channel and porous, e.g., microporous or nanoporous, membrane to provide a dispersion of lipid, protein and/or polymer shelled nanobubbles and/or microbubbles.

24-50. (canceled)
